(19) United States
(12) Patent Application Publication
(10) Pub. No.: US 2011/0070184 A1
(43) Pub. Date: Mar. 24, 2011

(54) METHODS AND COMPOSITIONS FOR TREATING ATHEROSCLEROSIS AND RELATED CONDIDTIONS

(75) Inventors: Jürgen Bernhagen, Aachen (DE); Christian Weber, Aachen (DE); Alma Zernecke, Aachen (DE)

(73) Assignee: CAROLUS THERPEUTICS, INC., San Diego, CA (US)

(21) Appl. No.: 12/918,960

(22) PCT Filed: Mar. 24, 2008
(86) PCT No.: PCT/US08/58048

§ 371 Date: Dec. 3, 2010

(30) Foreign Application Priority Data
Mar. 24, 2008 (US) PCT/US2008/058048

(51) Int. Cl. A61K 38/19; A61K 39/395; A61K 38/17; C07K 16/24; C07K 14/52; C07K 14/715; C07K 14/435; A61P 9/10

(52) U.S. Cl. 424/85.1; 424/158.1; 424/136.1; 424/133.1; 514/1.1; 530/389.2; 530/387.3; 530/351; 530/350

(57) ABSTRACT

Described herein are methods for treating or preventing a atherosclerosis by inhibiting the activation or binding of macrophage migration inhibitory factor to CXCR2 and CXCR4. Such inhibition is achieved by administration of either a single agent or a combination of agents. Also described herein are agents that inhibit MIF-binding or MIF-activation of CXCR2 or CXCR4. Further described are pharmaceutical compositions comprising such agents and their use for treating or preventing atherosclerosis.

### BACKGROUND

Atherosclerosis is an inflammatory condition in which there is a build-up of lipid-rich plaques within the walls of large arteries. Atherosclerosis and its associated pathology, e.g., atherosclerotic coronary artery disease, can lead to stroke and myocardial infarction and has almost invariably been the number one killer in the United States on an annual basis. Atherosclerosis is a multifactoral disease stemming from many different genetic and environmental factors and is the primary disease of the coronary arteries. Genetics, diabetes, hypercholesterolemia, hypertension, obesity, smoking, and physical inactivity are all known risk factors for the disease. Although atherosclerosis frequently remains clinically silent in its early stages and is often considered to be a disease associated with the later decades of life, the condition is evident at post-mortem examination even among individuals in their teens and twenties.

### SUMMARY OF THE INVENTION

Described herein are methods for treating or preventing atherosclerosis or otherwise alleviating any of the symptoms or pathologies associated with atherosclerosis. In one embodiment are such treatment or prevention methods comprising (a) administering an agent or agents that inhibits MIF- (macrophage migration inhibitory factor) based binding of CXCR2 and CXCR4; (b) administering an agent or agents that inhibits MIF-based activation of CXCR2 and CXCR4; (c) administering an agent or agents that inhibits MIF-based binding of CXCR2 and MIF-based activation of CXCR4; or (d) administering an agent or agents that inhibits MIF-based binding of CXCR4 and MIF-based activation of CXCR2. In a further embodiment, an agent is administered that inhibits MIF-based activation or MIF-based binding of CD74. In any of the aforementioned embodiments, a single agent is administered that to achieve the desired inhibitions. In an alternative embodiment, multiple different agents are administered (simultaneously or sequentially) to achieve the desired inhibitions.

Also described herein are uses of such agents for the formation of at least one medicament to treat or prevent atherosclerosis or otherwise alleviate any of the symptoms or pathologies associated with atherosclerosis.

Also described herein are pharmaceutical compositions of such agents for treating or preventing atherosclerosis or otherwise alleviating any of the symptoms or pathologies associated with atherosclerosis.

### CERTAIN DEFINITIONS

Unless indicated otherwise, the following terms have the following meanings when used herein and in the appended claims.

As used herein the term “treatment” or “treating” includes achieving a therapeutic benefit and/or a prophylactic benefit. By therapeutic benefit is meant eradication or amelioration of the underlying disorder or condition being treated. For example, in an individual with atherosclerosis, therapeutic benefit includes partial or complete halting of the progression of the disorder, or partial or complete reversal of the disorder. Also, a therapeutic benefit is achieved with the eradication or amelioration of one or more of the physiological or psychological symptoms associated with the underlying condition such that an improvement is observed in the patient, notwithstanding the fact that the patient is still affected by the condition. A prophylactic benefit of treatment includes prevention of a condition, retarding the progress of a condition, or decreasing the likelihood of occurrence of a condition. As used herein, “treating” or “treatment” includes prophylaxis.

As used herein, the phrase “biologically active” refers to a characteristic of any substance that has activity in a biological system and/or organism. For instance a substance is considered to be biologically active when that substance, when administered to an organism, has a biological effect on that organism. In particular embodiments, where a protein or polypeptide is biologically active, a portion of that protein or polypeptide that shares at least one biological activity of the protein or polypeptide is typically referred to as a “biologically active” portion.

As used herein, the term “effective amount” is an amount, which when administered, is sufficient to effect beneficial or desired results, such as beneficial or desired clinical results. An effective amount is also an amount that produces a prophylactic effect, e.g., an amount that delays, reduces, or eliminates the appearance of a pathological or undesired condition. Such conditions include, but are not limited to atherosclerosis. An effective amount is optionally administered in one or more administrations. In terms of treatment, an “effective amount” of a composition described herein is an amount that is sufficient to palliate, ameliorate, stabilize, reverse or slow the progression of an inflammatory condition. An “effective amount” includes any inhibitor of MIF or inhibitor of a MIF receptor used alone or in conjunction with one or more agents used to treat a disease or disorder. An “effective amount” of a therapeutic agent as described herein will be determined by a patient's attending physician or other medical care provider. Factors which influence what a therapeutically effective amount will be include, the pharmacokinetic profile of any inhibitor of MIF or inhibitor of a MIF receptor, age, physical condition, existence of other disease states, and nutritional status of the individual being treated. Additionally, other medication the patient is receiving, e.g. a cholesterol lowering agents, will typically affect the determination of the therapeutically effective amount of the therapeutic antibody to be administered.

As used herein, “expression” refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; (4) post-translational modification of a polypeptide or protein; (5) presentation of a polypeptide or protein on the cell surface; (6) secretion or release of a polypeptide or protein from a cell.

As used herein the terms “MIF”, “MIF polypeptide” or “MIF protein” are used interchangeably and refer to macrophage migration inhibitory factor GenBank Accession Numbers AAP36881 and CAG46452). Synonyms of MIF include, but are not limited to MMIF, Phenylpyruvate tautomerase, Glycosylation-inhibiting factor, GIF, GLIF and EC 5.3.2.1. In some embodiments, a MIF polypeptide comprises an amino acid sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers AAP36881 and CAG46452.

In some embodiments, a MIF gene comprises a nucleotide sequence that is at least 70% to 100% identical, e.g., at least 75%, 80%, B. %, 86%, 87%, 88%, 90%, 91%, 92%, 94%, 95%, 96%, 97%, 98%, or any other percent from about 70% to about 100% identical to sequences of GenBank Accession Number NM_{—}002415.

To determine the percent homology of two amino acid sequences or of two nucleic acids, the sequences are aligned for optimal comparison purposes (e.g., gaps are introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent homology between the two sequences is a function of the number of identical positions shared by the sequences (i.e., % identity=# of identical positions/total # of positions (e.g., overlapping positions)×100). In some embodiments the two sequences are the same length.

To determine percent homology between two sequences, the algorithm of Karlin and Altschul (1990) Proc. Natl. Acad. Sci. USA 87:2264-2268, modified as in Karlin and Altschul (1993) Proc. Natl. Acad. Sci. USA 90:5873-5877 is used. Such an algorithm is incorporated into the NBLAST and XBLAST programs of Altschul, et al. (1990) J. Mol. Biol. 215:403-410. BLAST nucleotide searches are performed with the NBLAST program, score=100, wordlength=12 to obtain nucleotide sequences homologous to a nucleic acid molecules described or disclose herein. BLAST protein searches are performed with the XBLAST program, score=50, wordlength=3. To obtain gapped alignments for comparison purposes, Gapped BLAST is utilized as described in Altschul et al. (1997) Nucleic Acids Res. 25:3389-3402. When utilizing BLAST and Gapped BLAST programs, the default parameters of the respective programs (e.g., XBLAST and NBLAST) are used. See the website of the National Center for Biotechnology Information for further details (on the world wide web at ncbi.nlm.nih.gov). Proteins suitable for use in the methods described herein also includes proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein described herein. In other embodiments, the altered amino acid sequence is at least 75% identical, e.g., 77%, 80%, 82%, 85%, 88%, 90%, 92%, 95%, 97%, 98%, 99%, or 100% identical to the amino acid sequence of any protein inhibitor described herein. Such sequence-variant proteins are suitable for the methods described herein as long as the altered amino acid sequence retains sufficient biological activity to be functional in the compositions and methods described herein. Where amino acid substitutions are made, the substitutions should be conservative amino acid substitutions. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al (1992), Proc. Natl Acad. Sci. USA, 89:10915-10919). Accordingly, the BLOSUM62 substitution frequencies are used to define conservative amino acid substitutions that, in some embodiments, are introduced into the amino acid sequences described or disclosed herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

As used herein, the term “receptor activation” or the term “activation” when referring to a receptor, unless otherwise specified, means that a receptor is in a state competent to effect signal transduction. Examples of signal transduction events initiated by receptor activation include, but are not limited to, any of the following: heterodimerization, G-protein activation, calcium influx, caspase activation, protease activation, increased protein kinase activity, or transcriptional activation or repression.

As used herein, the term “inhibits” or “inhibition” unless otherwise specified means partial or complete inhibition.

As used herein, a “MIF inhibitor” refers to any antibody composition or peptide mimetic of MIF that directly or indirectly decreases MIF activity. In some embodiments, MIF inhibitors decrease MIF activity by reducing and/or abolishing binding of endogenous MIF to at least one of its natural binding partners (e.g., CD74, CXCR2 and CXCR4) or MIF-dependent receptor activation of at least one of its natural binding partners (e.g., CD74, CXCR2 and CXCR4) as measured using standard methods. Thus, in some embodiments, binding between MIF and at least one of its natural binding partners is stronger in the absence of the inhibitor than in its presence.

The term “MIF receptor inhibitor” is used herein for the purposes of the specifications and claims, to mean any antibody composition or antibody disclosed herein that inhibits the activation of CD74, CXCR2 or CXCR4. The term “MIF Receptor Inhibitor” includes, but is not limited to compositions of MIF inhibitors, MIF analogs, peptide mimetics of MIF, MIF antagonists, anti-MIF antibodies or antigen binding fragments thereof, anti-CXCR2 antibodies or antigen binding fragments thereof, the CXCR4 antagonists AMD3465, anti-CXCR4 antibodies or antigen binding fragments thereof, anti-CD74 antibodies or fragments thereof, antibodies that inhibit CD74-mediated activation of a G-protein coupled receptor and antibodies that inhibit CD74-mediated activation of CXCR2 or any combination thereof.

The term “peptide mimetic”, “mimetic peptide” and “analog” are used herein interchangeably for the purposes of the specifications and claims, to mean a peptide that mimics part or all of the bioactivity of an endogenous protein ligand. Non-limiting examples of peptide mimetic can be found in DE19964386, U.S. Pat. No. 7,303,885, and EP1334195, all of which are incorporated by reference in their entirety.

In one embodiment, peptide mimetics are modeled after a specific ligand and display an altered peptide backbone, altered amino acids and/or an altered primary amino acid sequence when compared to the ligand of which is was designed to mimic. Peptide mimetics are typically designed to impart selective receptor binding and selective receptor activation properties. In some embodiments, a peptide mimetic of MIF, through competitive binding to CXCR2, inhibits the bioactivity of endogenous MIF to CXCR2. In some embodiments, a peptide mimetic of MIF selectively inhibits the binding of endogenous MIF to CXCR2 and CXCR4, but not CD74. In some embodiments a peptide mimetic of MIF binds a receptor and imparts a partial signal. In some embodiments, for example, a peptide mimetic of MIF binds CD74 and activates ERK-MAP kinases but inhibits activation of GPCRs and CXCR2.

The design of peptide mimetics is possible because the interaction of a protein ligand with its receptor often takes place over a relatively large interface. Human growth hormone, for example, binds to its receptor using only a few key residues that contribute to most of the binding energy (Clackson, T. et al., Science 267:383-386 (1995)). The bulk of the remaining growth hormone ligand serves only to display the key binding residues in the correct topology.

Peptide mimetics are developed using, for example, computerized molecular modeling. Peptide mimetics are designed to include structures having one or more peptide linkages optionally replaced by a linkage selected from the group consisting of: —CH₂NH—, —CH₂S—, —CH₂—CH₂—, —CH═CH-(cis and trans), —CH═CF-(trans), —CoCH₂—, —CH(OH)CH₂—, and —CH₂SO—, by methods well known in the art. In some embodiments such peptide mimetics have greater chemical stability, enhanced pharmacological properties (half-life, absorption, potency, efficacy, etc.), altered specificity (e.g., a broad-spectrum of biological activities), reduced antigenicity, and are more economically prepared. In some embodiments peptide mimetics include covalent attachment of one or more labels or conjugates, directly or through a spacer (e.g., an amide group), to non-interfering positions(s) on the analog that are predicted by quantitative structure-activity data and/or molecular modeling. Such non-interfering positions generally are positions that do not form direct contacts with the receptor(s) to which the peptide mimetic binds to produce the therapeutic effect. In some embodiments systematic substitution of one or more amino acids of a consensus sequence with a D-amino acid of the same type (e.g., D-lysine in place of L-lysine) are used to generate more stable peptides with desired properties.

Phage display peptide libraries have emerged as a powerful technique in generating peptide mimetics (Scott, J. K. et al. (1990) Science 249:386; Devlin, J. J. et al. (1990) Science 249:404; U.S. Pat. No. 5,223,409, U.S. Pat. No. 5,733,731; U.S. Pat. No. 5,498,530; U.S. Pat. No. 5,432,018;U.S. Pat. No. 5,338,665;U.S. Pat. No. 5,922,545; WO 96/40987 and WO 98/15833 (each of which is incorporated by reference in its entirety). In such libraries, random peptide sequences are displayed by fusion with coat proteins of filamentous phage. Typically, the displayed peptides are affinity-eluted against an antibody-immobilized extracellular domain of a receptor. In some embodiments peptide mimetics of MIF that bind CXCR2 or CXCR4 are isolated by biopanning (Nowakowski, G. S, et al. (2004) Stem Cells 22:1030-1038). In some embodiments whole cells expressing CXCR2 or CXCR4 are used to screen the library utilizing FACs to isolate phage bound cells. The retained phages are enriched by successive rounds of biopanning and repropagation. The best binding peptides are sequenced to identify key residues within one or more structurally related families of peptides. The peptide sequences also suggest which residues to replace by alanine scanning or by mutagenesis at the DNA level. In some embodiments mutagenesis libraries are created and screened to further optimize the sequence of the best binders. Lowman (1997) Ann. Rev. Biophys. Biomol. Struct. 26:401-24.

In some embodiments structural analysis of protein-protein interaction is used to suggest peptides that mimic the binding activity of endogenous protein ligands. In some embodiments the crystal structure resulting from such an analysis suggests the identity and relative orientation of critical residues of the endogenous protein ligand, from which a peptide is designed. See, e.g., Takasaki, et al. (1997) Nature Biotech, 15: 1266-70. In some embodiments these analytical methods are used to investigate the interaction between a receptor protein and peptides selected by phage display, and suggest further modification of the peptides to increase binding affinity.

The term “compound” is used herein, for purposes of the specification and claims, to mean any peptide, protein, antibody, antigen binding fragment, small molecule or combination thereof that is formulated for administration into a patient.

The term “agent” is used herein, for purposes of the specification and claims, to mean any peptide, protein, antibody, antigen binding fragment, small molecule or combination thereof that is formulated for administration into a patient. As used herein the term “agent” is synonymous with the term “compound”.

The term “small molecule” (also called a “small compound”) is used herein, for purposes of the specification and claims, to mean a compound having a molecular weight of 1000 or less; for example, an organic compound, an inorganic compound or a derivative thereof that is usable as a pharmaceutical. A “small molecule” more specifically refers to a compound produced by making use of a method of organic synthesis, a naturally occurring compound or a derivative thereof, and may comprise various metals or salts. Small compounds can be commercially available if they are known compounds, or can be obtained via steps such as of collection, production and purification according to various publications

As used herein, the terms “antibody” and “antibodies” refer to monoclonal antibodies, polyclonal antibodies, bi-specific antibodies, multispecific antibodies, grafted antibodies, human antibodies, humanized antibodies, synthetic antibodies, chimeric antibodies, camelized antibodies, single-chain Fvs (scFv), single chain antibodies, Fab fragments, F(ab) fragments, disulfide-linked Fvs (sdFv), intrabodies, and anti-idiotypic (anti-Id) antibodies and antigen-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG₁, IgG₂, IgG₃, IgG₄, IgA₁ and IgA₂) or subclass. The terms “antibody” and immunoglobulin are used interchangeably in the broadest sense. The subunit structures and three-dimensional configurations of the different classes of immunoglobulins are well known in the art. The term antibodies includes for example anti-MIF, anti-CXCR2, anti-CXCR4, anti-CD44, anti-CD74 and anti-GPCR antibodies of the invention. In some embodiments an antibody is part of a larger fusion molecule, formed by covalent or non-covalent association of the antibody with one or more other proteins or peptides.

As used herein, the term “derivative” in the context of a polypeptide or protein, e.g. an antibody, refers to a polypeptide or protein that comprises an amino acid sequence which has been altered by the introduction of amino acid residue substitutions, deletions or additions. The term “derivative” as used herein also refers to a polypeptide or protein which has been modified, i.e., by the covalent attachment of any type of molecule to the antibody. For example, in some embodiments a polypeptide or protein is modified, e.g., by glycosylation, acetylation, pegylation, phosphorylation, amidation, derivatization by known protecting/blocking groups, proteolytic cleavage, linkage to a cellular ligand or other protein, etc. In some embodiments derivatives, polypeptides or proteins are produced by chemical modifications using techniques known to those of skill in the art, including, but not limited to specific chemical cleavage, acetylation, formylation, metabolic synthesis of tunicamycin, etc. In some embodiments a derivative, a polypeptide or protein possesses a similar or identical function as the polypeptide or protein from which it was derived.

The terms “full length antibody”, “intact antibody” and “whole antibody” are used herein interchangeably, to refer to an antibody in its substantially intact form, and not antibody fragments as defined below. These terms particularly refer to an antibody with heavy chains contains Fc regions. In some embodiments an antibody variant of the invention is a full length antibody. In some embodiments the full length antibody is human, humanized, chimeric, and/or affinity matured.

An “affinity matured” antibody is one having one or more alteration in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by known procedures. See, for example, Marks et al., (1992) Biotechnology 10:779-783 that describes affinity maturation by variable heavy chain (VH) and variable light chain (VL) domain shuffling. Random mutagenesis of CDR and/or framework residues is described in: Barbas, et al. (1994) Proc. Nat. Acad. Sci, USA 91:3809-3813; Shier et al., (1995) Gene 169:147-155; Yelton et al., 1995, J. Immunol. 155:1994-2004; Jackson et al., 1995, J. Immunol. 154(7):3310-9; and Hawkins et al, (19920, J. Mol. Biol. 226:889-896, for example.

An “antagonist antibody” is an antibody that binds and inactivates an antigen, such as a receptor. In some embodiments the inactivation of an antigen receptor is partial. In some embodiments an antagonist antibody binds its target receptor and prevents a specific ligand from binding, thus blocking the signaling activity of that specific ligand. Therefore, in some embodiments, antagonist antibodies that bind to different epitopes on the antigen receptor block the activity and binding of specific ligands. For example, in some embodiments an antibody that binds a specific epitope on CXCR4 prevents binding MIF, yet allows binding of CXCL12. In some embodiments antagonist antibodies that bind different epitopes on the same antigen receptor effectively block the activity of a sub-set of ligands that bind to that receptor while not effecting the binding or activity of other ligands that bind the same receptor. In some embodiments antagonist antibodies block receptor activity by sterically hindering the formation of active receptor complexes. For example, in some embodiments, an antagonist antibody to a receptor prevents the interaction of the receptor with a co-signaling molecule, such as a GPCR. Likewise, in some embodiments, an antagonist antibody specifically binds a ligand and prevents its binding or activation of a receptor. In some embodiments an antagonist antibody specifically binds a particular epitope of a ligand and prevents its binding or activation of a specific receptor while allowing activation of another receptor for that ligand. For example, in some embodiments an anti-MIF antibody binds a specific epitope on MIF and prevents MIF binding to CXCR2 while allowing MIF to bind CD74.

The terms “binding fragment”, “antibody fragment” or “antigen binding fragment” are used herein, for purposes of the specification and claims, to mean a portion or fragment of an intact antibody molecule, preferably wherein the fragment retains antigen-binding function. Examples of antibody fragments include Fab, Fab′, F(ab′)₂, Fd, Fd′ and Fv fragments, diabodies, linear antibodies (Zapata et al. (1995) Protein Eng. 10: 1057), single-chain antibody molecules, single-chain binding polypeptides, scFv, bivalaent scFv, tetravalent scFv, and bispecific or multispecific antibodies formed from antibody fragments.

“Fab” fragments are typically produced by papain digestion of antibodies resulting in the production of two identical antigen-binding fragments, each with a single antigen-binding site and a residual “Fc” fragment. Pepsin treatment yields a F(ab′)2 fragment that has two antigen-combining sites capable of cross-linking antigen. An “Fv” is the minimum antibody fragment that contains a complete antigen recognition and binding site. In a two-chain Fv species, this region consists of a dimer of one heavy- and one light-chain variable domain in tight, non-covalent association. In a single-chain Fv (scFv) species, one heavy- and one light-chain variable domain are covalently linked by a flexible peptide linker such that the light and heavy chains associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although usually at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy-chain C_{H}1 domain including one or more cysteines from the antibody hinge region. Fab′-SH is the designation herein for Fab′ in which the cysteine residue(s) of the constant domains bear a free thiol group. F(ab)₂ antibody fragments originally were produced as pairs of Fab′ fragments that have hinge cysteines between them. Other chemical couplings of antibody fragments are also known. Methods for producing the various fragments from monoclonal Abs are well known to those skilled in the art (see, e.g., Pluckthum, 1992, Immunol. Rev. 130:152-188).

The term “monoclonal antibody” as used herein refers to an antibody obtained from a population of substantially homogeneous antibodies, i.e., the individual antibodies comprising the population are identical except for possible naturally occurring mutations that are present in minor amounts. In some embodiments monoclonal antibodies are made, for example, by the hybridoma method first described by Kohler and Milstein (1975) Nature 256:495, or are made by recombinant methods, e.g., as described in U.S. Pat. No. 4,816,567. In some embodiments monoclonal antibodies are isolated from phage antibody libraries using the techniques described in Clackson et al., Nature 352:624-628 (1991), as well as in Marks et al., J. Mol. Biol. 222:581-597 (1991).

The antibodies herein include monoclonal, polyclonal, recombinant, chimeric, humanized, bi-specific, grafted, human, and fragments thereof including antibodies altered by any means to be less immunogenic in humans. Thus, for example, the monoclonal antibodies and fragments, etc., herein include “chimeric” antibodies and “humanized” antibodies. In general, chimeric antibodies include a portion of the heavy and/or light chain that is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, so long as they exhibit the desired biological activity (U.S. Pat. No. 4,816,567); Morrison et al. Proc. Natl Acad. Sci. 81:6851-6855 (1984). For example in some embodiments a chimeric antibody contains variable regions derived from a mouse and constant regions derived from human in which the constant region contains sequences homologous to both human IgG2 and human IgG4. Numerous methods for preparing “chimeric” antibodies, etc., are known in the art. “Humanized” forms of non-human (e.g., murine) antibodies or fragments are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include, grafted antibodies or CDR grafted antibodies wherein part or all of the amino acid sequence of one or more complementarity determining regions (CDRs) derived from a non-human animal antibody is grafted to an appropriate position of a human antibody while maintaining the desired binding specificity and/or affinity of the original non-human antibody. In some embodiments, corresponding non-human residues replace Fv framework residues of the human immunoglobulin. In some embodiments humanized antibodies comprise residues that are found neither in the recipient antibody nor in the imported CDR or framework sequences. These modifications are made to further refine and optimize antibody performance. In some embodiments, the humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. For further details, see, e.g.: Jones et al., Nature 321: 522-525 (1986); Reichmann et al., Nature 332: 323-329 (1988) and Presta, Curr. Op. Struct. Biol. 2: 593-596 (1992). Numerous methods for “humanizing” antibodies, etc., are known in the art.

As used herein, the term “epitope” refers to a fragment of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

The phrase “specifically binds” when referring to the interaction between an antibody or other binding molecule and a protein or polypeptide or epitope, typically refers to an antibody or other binding molecule that recognizes and detectably binds with high affinity to the target of interest. Preferably, under designated or physiological conditions, the specified antibodies or binding molecules bind to a particular polypeptide, protein or epitope yet does not bind in a significant or undesirable amount to other molecules present in a sample. In other words the specified antibody or binding molecule does not undesirably cross-react with non-target antigens and/or epitopes. Further it is understood to one skilled in the art, that in some embodiments, an antibody that specifically binds, binds through the variable domain or the constant domain of the antibody. For the antibody that specifically binds through its variable domain, it is understood to one skilled in the art that it is not aggregated, i.e., is monomeric. A variety of immunoassay formats are used to select antibodies or other binding molecule that are immunoreactive with a particular polypeptide and have a desired specificity. For example, solid-phase ELISA immunoassays, BIAcore, flow cytometry and radioimmunoassays are routinely used to select monoclonal antibodies having a desired immunoreactivity and specificity. See, Harlow, 1988, ANTIBODIES, A LABORATORY MANUAL, Cold Spring Harbor Publications, New York (hereinafter, “Harlow”), for a description of immunoassay formats and conditions that are used to determine or assess immunoreactivity and specificity. “Selective binding”, “selectivity”, and the like refer the preference of a antibody to interact with one molecule as compared to another. Preferably, interactions between antibodies, particularly modulators, and proteins are both specific and selective. Note that in some embodiments a small antibody is designed to “specifically bind” and “selectively bind” two distinct, yet similar targets without binding to other undesirable targets. For example, a protein kinase C (PKC) inhibitor can selectively bind and inhibit PKCα and PKCβ without binding or inhibiting PKCγ.

As used herein, the term “endogenous” in the context of a cellular protein refers to protein naturally occurring and/or expressed by the cell in the absence of recombinant manipulation; accordingly, the terms “endogenously expressed protein” or “endogenous protein” excludes cellular proteins expressed by means of recombinant technology.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, the terms “prophylactic treatment” refer to the methods of the disclosed invention and the administration of any antibody(s) that is used in the prevention of a disorder, or prevention of recurrence or spread of a disorder.

As used herein, the term “stable plaque phenotype” refers to a decrease of macrophage infiltration in the intima and neointima with a concominant increase in small muscle cell and collagen content when a comparison is made between a time point before treatment and some time point after treatment. In some embodiments the term refers to a decrease in macrophage content of an atherosclerotic plaque when a comparison is made between a time point before treatment and some time point after treatment. In some embodiments the term refers to a decrease in T-cell content of an atherosclerotic plaque when a comparison is made between a time point before treatment and some time point after treatment. In some embodiments the term refers to an increase in smooth muscle cell content of an atherosclerotic plaque when a comparison is made between a time point before treatment and some time point after treatment. In some embodiments the term refers to an increase in collagen content of an atherosclerotic plaque when a comparison is made between a time point before treatment and some time point after treatment. In some embodiments a decrease of macrophage content means a decrease of between 5% and 100%, wherein a decrease of 100% means that there are no detectable macrophages in the sample after treatment. In some embodiments a decrease of macrophage content means a decrease of between 20% and 100%. In some embodiments a decrease of T-cell content means a decrease of between 5% and 100%, wherein a decrease of 100% means that there are no detectable T-cells in the sample after treatment. In some embodiments an increase of smooth muscle cell content means an increase of between 2% and 200%, wherein a 200% increase means that there are twice as many smooth muscle cells detected in a sample after treatment. In some embodiments an increase of smooth muscle cell content means an increase of between 2% and 1000%. In some embodiments an increase in collagen content means an increase of between 2% and 200%, wherein a 200% increase means that there is twice as much collagen in a sample after treatment. In some embodiments an increase in collagen content means an increase of between 2% and 1000%.

As used herein, the term “regression of pre-existing atherosclerotic plaques” refers to a regression in the pathology of an atherosclerotic plaque. The pathology of an atherosclerotic plaque is defined by the number of infiltrating macrophages, the number of infiltrating T-cells, the collagen content of the plaque, the number of smooth muscle cells, the type of atherosclerotic lesion (e.g. Type I through Type VIII) and the content of the atherosclerotic lesion. A regression in the pathology of an atherosclerotic plaque is indicated by at least one of the following: 1) a decrease in the number of infiltrating macrophages; 2) a decrease in the number of infiltrating T-cells; 3) an increase in the number of smooth muscle cells; 4) a decrease in the collagen content of the plaque; 5) a re-classification of the lesion to a lower Type (e.g. from Type VI to Type V); a decrease in plaque size; a decrease in intima thickness; and an increase in lumen size.

As used herein, the terms “manage,” “managing” and “management” refer to the beneficial effects that a subject derives from administration of a prophylactic or therapeutic antibody, which does not result in a cure of the disease. In certain embodiments, a subject is administered one or more prophylactic or therapeutic antibodies to “manage” a disease so as to prevent the progression or worsening of the disease.

As used herein, the terms “prevent”, “preventing” and “prevention” refer to the prevention of the occurrence and/or recurrence or onset of one or more symptoms of a disorder in a subject resulting from the administration of a prophylactic or therapeutic antibody.

As used herein, the term “in combination” refers to the use of more than one compound. The use of the term “in combination” does not restrict the order in which the compounds are administered to a patient in need thereof. In some embodiments a compound is administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second compound to a patient in need thereof. In some embodiments the compounds are administered simultaneously, as a mixture within the same formulation.

The terms “polypeptide”, peptide” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to antibodies that have the same basic chemical structure as a naturally occurring amino acid, i.e., an α carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions are achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid is one that is separated from the nucleic acids that normally flank it or other nucleic acids or components (proteins, lipids, etc. . . . ) in a sample. In another example, a polypeptide is purified if it is substantially removed from or concentrated in its natural environment. Methods for purification and isolation of nucleic acids and proteins are documented methodologies.

A “subject” or an “individual,” as used herein, is an animal, for example, a human patient. In some embodiments a “subject” or an “individual” is a human. In some embodiments, the subject suffers from an inflammatory condition or atherosclerosis.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. MIF-triggered mononuclear cell arrest is mediated by CXCR2/CXCR4 and CD74. Human aortic endothelial cells (HAoECs), CHO cells stably expressing ICAM-1 (CHO/ICAM-1) and mouse microvascular endothelial cells (SVECs) were preincubated with or without MIF (together with antibody to MIF, antibodies to CXCL1 and CXCL8, or isotype control), CXCL8, CXCL10 or CXCL12 for 2 h as indicated. Mononuclear cells were pretreated with antibodies to CXCR1, CXCR2, β₂, CXCR4, CD74, or isotype controls for 30 min, or pertussis toxin (PTX) for 2 h as indicated. (a) HAoECs were perfused with primary human monocytes. (b) Immunofluorescence using antibody to MIF revealed surface presentation of MIF (green) on HAoECs and CHO/ICAM-1 cells after pretreatment for 2 h, but not 30 min (not shown); in contrast, MIF was absent in buffer-treated cells (control). Scale bar, 100 μm. (c,d) CHO/ICAM-1 cells were perfused with MonoMac6 cells. (e) HAoECs were perfused with T cells. (f,g) CHO/ICAM-1 cells were perfused with Jurkat T cells (f), and with Jurkat CXCR2 transfectants or vector controls (g). In c, d, f and g, background binding to vector-transfected CHO cells was subtracted. (h) Mouse SVECs were perfused with L1.2 transfectants stably expressing CXCR1, CXCR2 or CXCR3, and with controls expressing only endogenous CXCR4, in the presence of the CXCR4 antagonist AMD3465. Arrest is quantified as cells/mm² or as percentage of control cell adhesion. Data in a and c-g represent mean±s.d. of 3-8 independent experiments; data in h are results from one representative experiment of four experiments.

FIG. 2. MIF-triggered mononuclear cell chemotaxis is mediated by CXCR2/CXCR4 and CD74. Primary human monocytes (a-e), CD3⁺ T cells (f) and neutrophils (g,h) were subjected to transmigration analysis in the presence or absence of MIF. CCL2 (a), CXCL8 (a,g,h) and CXCL12 (f) served as positive controls or were used to test desensitization by MIF (or by CXCL8, h). The chemotactic effects of MIF, CCL2 and CXCL8 on monocytes (a) or of MIF on neutrophils (g) followed bell-shaped dose-response curves. MIF-triggered chemotaxis of monocytes was abrogated by an antibody to MIF, boiling (b), or by MIF at indicated concentrations (in the top chamber; c). (d) MIF-triggered chemotaxis was mediated by G_{αi}/phosphoinositide-3-kinase signaling, as evidenced by treatment with pertussis toxin components A and B (PTX A+B), PTX component B alone or Ly294002. (e) MIF-mediated monocyte chemotaxis was blocked by antibodies to CD74 or CXCR1/CXCR2. (f) T-cell chemotaxis induced by MIF was blocked by antibodies to MIF and CXCR4. (g) Neutrophil chemotaxis induced by MIF. (h) MIF-induced versus CXCL8-induced neutrophil chemotaxis, effects of antibodies to CXCR2 or CXCR1, and desensitization of CXCL8 by MIF. Data in a and f-h are expressed as chemotactic index; data in c are expressed as percent of control; and data in b,d and e as percent of input. Data represent mean±s.d. of 4-10 independent experiments, except for panels a,c and g, boiled MIF in b, and the antibody-alone controls in b and e, which are means of 2 independent experiments.

FIG. 3. MIF triggers rapid integrin activation and calcium signaling. (a) Human aortic endothelial cells were stimulated with MIF or TNF-α for 2 h. CXCL1 and CXCL8 mRNAs were analyzed by real-time PCR and normalized to control. Supernatant-derived CXCL8 was assessed by ELISA (n=3 independent experiments performed in duplicate). (b) MonoMac6 cells were directly stimulated with MIF or CXCL8 for 1 min and perfused on CHO-ICAM-1 cells for 5 min (mean±s.d. of 8 independent experiments). (c) MonoMac6 cells were stimulated with MIF for the indicated times. LFA-1 activation (detected by the 327C antibody) was monitored by FACSAria, and expressed as the increase in mean fluorescence intensity (MFI). (d) As in c but for primary monocytes; data are expressed relative to maximal activation with Mg²⁺/EGTA. (e) MonoMac6 cells were pretreated with antibodies to α₄ integrin, CD74 or CXCR2, stimulated with MIF for 1 min, perfused on VCAM-1.Fc for 5 min. Adhesion is expressed as a percentage of controls. Arrest data in c-e represent mean±s.d. of 5 independent experiments. (f) Calcium transients in Fluo-4 AM-labeled neutrophils were stimulated with MIF, CXCL8 or CXCL7. Calcium-derived MFI was recorded by FACSAria for 0-240 s. For desensitization, stimuli were added 120 s before stimulation. Traces shown represent 4 independent experiments. (g) Dose-response curves of calcium-influx triggered by CXCL8, CXCL7 or MIF, at indicated concentrations, in L1.2-CXCR2 transfectants. Data are expressed as the difference between baseline and peak MFI (mean±s.d. of 4-8 independent experiments).

FIG. 4. MIF-interaction with CXCR2/CXCR4 and formation of CXCR2/CD74 complexes. HEK293-CXCR2 transfectants (a) or CXCR4-bearing Jurkat T-cells (c) were subjected to receptor binding assays, analyzing competition of [I¹²⁵]CXCL8 (a) or [I¹²⁵]CXCL12 (c) by MIF or cold cognate ligand (mean±s.d., n=6-10). (b) MIF- and CXCL8-induced CXCR2 internalization in HEK293-CXCR2 or RAW264.7-CXCR2 transfectants (inset shows representative histograms) as indicated; assessed by FACS analysis of surface CXCR2 expression (percentage of buffer (Con), mean±s.d., n=5). (d) MIF- and CXCL12-induced CXCR4 internalization in Jurkat T-cells as in b (mean±s.d., n=4-6). (e) Binding of fluorescein-MIF to HEK293-CXCR2 transfectants or vector controls analyzed by FACS. Inset shows binding of biotin-MIF to CXCR2 assessed by western blot using antibodies to CXCR2 after streptavidin (SAv) pull-down from HEK293-CXCR2 transfectants versus vector controls. (f) Colocalization of CXCR2 and CD74 (orange-yellow overlay) in RAW264.7-CXCR2 transfectants stained for CXCR2, CD74 and nuclei (Hoechst), analyzed by fluorescence microscopy (top) or confocal laser scanning microscopy (bottom). Scale bar, 10 μm. (g) Coimmunoprecipitation of CXCR2/CD74 complexes in CHAPSO-extracts of HEK293-CXCR2 transfectants expressing His-tagged CD74. Anti-His immunoprecipitation (IP) followed by anti-CXCR2 or anti-His-CD74 western blotting (WB; top) or anti-CXCR2 immunoprecipitation followed by anti-His-CD74 or anti-CXCR2 western blotting (bottom). Controls: lysates without immunoprecipitation or beads alone. (h) As in g for L1.2-CXCR2 transfectants. Anti-CXCR2 immunoprecipitation from L1.2-CXCR2 transfectants followed by anti-CD74 or anti-CXCR2 western blotting (top). Immunoprecipitation with isotype IgG or CXCR2-negative L1.2-cells (bottom) served as controls. Data represent 3 independent experiments (e-h).

FIG. 5. MIF-driven monocyte arrest in inflamed or atherosclerotic arteries involves CXCR2. (a) Monocyte arrest in carotid arteries from Apoe^{-/-} mice fed a western diet for 6 weeks. (b,c) Monocyte arrest in carotid arteries from Mif^{+/+} and Mif^{-/-} mice 4 h after intraperitoneal injection of TNF-α. (d-f) MonoMac6 cell arrest in carotid arteries from Mif^{+/+} Ldlr^{-/-} and Mif^{-/-} Ldlr^{-/-} mice fed a western diet for 6 weeks (n=3 each). In f, carotid arteries were loaded with MIF for 2 h before perfusion with MonoMac6 cells. After 10 min, adherent cells in 5-6 fields per carotid artery were counted. Data represent mean±s.d. of 3 independent experiments. (g,h) For intravital microscopy, Mif^{+/+} and Mif^{-/-} mice reconstituted with wild-type or Il8rb^{-/-} bone marrow (n=3 each) were stimulated by intraperitoneal injection of TNF-α for 4 h, and the accumulation of leukocytes labeled by intravenous injection of rhodamine G was studied after 30 min in carotid arteries in vivo. Scale bar, 50 μm. Data in g are expressed as mean±s.d. Representative segments are shown in h.

FIG. 6. MIF-induced atherogenic and microvascular inflammation through CXCR2 in vivo and effects of MIF blockade on plaque regression. (a) Monocyte adhesion to the lumen in vivo and lesional macrophage content in native aortic roots were determined in Mif^{+/+} Ldlr^{-/-} and Mif^{-/-} Ldlr^{-/-} mice (n=4) fed a chow diet for 30 weeks. Representative images are shown. Arrows indicate monocytes adherent to the lumina' surface. Scale bar, 100 μm. (b,c) Exposure to MIF induced CXCR2-dependent leukocyte recruitment in vivo. Following intrascrotal injection of MIF, the cremasteric microvasculature was visualized by intravital microscopy. Pretreatment with blocking CXCR2 antibody abrogated adhesion and emigration, as compared to IgG control (n=4). (d) Intraperitoneal injection of MIF or vehicle elicited neutrophil recruitment in wild-type mice (n=3) reconstituted with wild-type, but not Il8rb^{-/-}, bone marrow. (e-h) Blocking MIF but not CXCL1 or CXCL12 resulted in regression and stabilization of advanced atherosclerotic plaques. Apoe^{-/-} mice received a high-fat diet for 12 weeks and were subsequently treated with antibodies to MIF, CXCL1 or CXCL12, or with vehicle (control) for an additional 4 weeks of (n=6-10 mice). Plaques in the aortic root were stained using Oil-Red-O. Representative images are shown in e (scale bars, 500 μm). Data in f represent plaque area at baseline (12 weeks) and after 16 weeks. The relative content of MOMA-2⁺ macrophages is shown in g and the number of CD3⁺ T cells per section in h. Data represent mean±s.d.

FIG. 7 is an illustrative representation of the structural homology between the CXCL8 dimer and the MIF monomer. Ribbon representations of an interleukin-8/CXCL8 dimer (blue) and a monomer of macrophage migration inhibitory factor (MIF, pink) with highlighted amino acid side chains glutamic acid E9 and arginine R11 in CXCL8 and aspartic acid D44 and arginine R11 in MIF. Ribbon images were created with Swiss PdbViewer. Shown in the white text box are amino acids 1-50 of CXCL8 and MIF and the complete amino acid sequence of human beta defensin 1 (hBD1) in single letter codes, revealing no significant homology at the level of primary structure. Numbering of the MIF sequence refers to the processed protein sequence, i.e. with the N-terminal methionine residue cleaved and numbering starting at the following proline (Prol) residue.

FIG. 8. (a) MIF-triggered arrest of monocytic MonoMac6 cells is mediated by CXCR2. Human aortic endothelial cells (HAoEC) were perfused with cells of the human monocytic MonoMac6 cell line. Adherent HAoEC monolayers were preincubated with or without recombinant MIF (50 ng/mL) for 2 h and perfused with MonoMac6 cells at a flow rate of 1.5 dyne/cm2 for 2 min. Immunofluorescence using a rabbit MIF antiserum revealed surface presentation of MIF on HAoEC (see also FIG. 1b). MIF-mediated arrest of MonoMac6 cells was inhibited by pretreatment with antibodies to MIF or CXCR2, or with pertussis toxin (PTX) as indicated. Monocyte arrest is expressed as cells/mm². Data represent means±SD of 4 independent experiments. (b) Monocytic cell arrest triggered by CXCL8 is dependent on CXCR2. CHO cells stably expressing ICAM-1 (CHO-ICAM-1) were preincubated with CXCL8 for 2 h and perfused with MonoMac6 cells at a flow rate of 1.5 dyne/cm2 for 2 min. While a blocking antibody to CXCR1 had no effect and blocking CXCR2 significantly but not fully attenuated the arrest functions of CXCL8, combining these antibodies completely and significantly inhibited monocytic cell arrest triggered by CXCL8 as compared with isotype control IgG. The data are means±SD of 4 independent experiments. (c) Adhesion of monocytic MonoMac6 cells to a CHO/ICAM-1 monolayer under flow conditions—‘antibody alone’ control incubations. Confluent CHO cells stably expressing human ICAM-1 were perfused with MonoMac6 in a parallel wall flow chamber. As shown, treatment of the MonoMac6 cells with antibodies against the chemokine receptors CXCR1 and CXCR2 did not result in a significant change in adherence properties. Similarly, the mAb against the MIF surface binding protein CD74 had no effect. After incubation with the respective antibody for 30 min, cells were resuspended in assay buffer and perfused over the CHO-ICAM-1 monolayers at 1.5 dynes/cm2. After 2 min of perfusion, firmly adhered cells were quantified in multiple fields. The Data are means±SD of 6-9 independent experiments and are expressed as percent control, i.e. untreated MonoMac6 cells. (d) MIF promotes chemotaxis of RAW 264.7 macrophages. The migratory effect of MIF on RAW macrophages is comparable to that obtained with Ccl2 and is blocked by an antibody to Cd74. RAW 264.7 cells (5×104) labeled with calcein-AM for 2 h in RPMI 1640/10% FCS were placed in the upper chamber of 8 μm pore Transwell filters, and allowed to migrate towards MIF (50 ng/mL) or Ccl2 (50 ng/mL) in the bottom chamber for 3 h. For blockade of Cd74, cells were pre-incubated with a antibody to murine Cd74 for 30 min. Migrated calcein AM-labeled macrophages were quantitated by automated fluorescent analysis of migrated calcein-positive cells using a Wallac Victor fluorescent plate reader at an excitation/emission wavelength of 485/535 nm. Data are means±SD of 4 independent experiments and are expressed as transmigration in relation to input. (e) Cross-desensitization of CXCL12-induced T-cell migration by MIF. The migratory effect of MIF on unstimulated primary human T cells is dose-dependent and chemotactic in nature, as shown by cross-desensitization of the CXCL 12-induced migration by MIF. Unstimulated primary CD3+ T-cells (5×104) labeled with calcein-AM for 2 h in RPMI 1640/10% FCS were placed in the upper chamber of 3 μm pore Transwell filters, and allowed to migrate towards MIF (10, 50 or 100 ng/mL) or CXCL12 (50 or 250 ng/mL) in the bottom chamber for 1.5 h. For heterologous desensitization of CXCL12-mediated migration by MIF, T-cells migrating towards CXCL12 (50 ng/mL) were pre-incubated with MIF (50 ng/mL) for 30 min. Migrated calcein AM-labeled T-cells were quantitated by automated fluorescent analysis of migrated calcein-positive cells using a Wallac Victor fluorescent plate reader at an excitation/emission wavelength of 485/535 nm. Data are means±SD of 4 independent experiments and are expressed as transmigration in relation to input. (f) Dependence on CXCR4 of MIF-triggered Jurkat T-cell arrest on immobilized VCAM-1. Jurkat T-cells treated with a blocking antibody against the CXCL12 receptor CXCR4 or with isotype-matched control IgG were perfused over immobilized VCAM-1.Fc after direct stimulation with MIF. Spontaneous cell arrest on VCAM-1.Fc was not affected by antibodies to CXCR4, whereas adhesion of MIF-stimulated cells was significantly inhibited by anti-CXCR4. Jurkat T-cells were directly stimulated with MIF and perfused over 35 mm dishes coated with VCAM-1.Fc at 1.5 dynes/mm2. After 2 min of perfusion, cell arrest was quantified in multiple fields. Data are expressed as % of control (i.e. untreated cells) and data represent means±SD of 4 independent experiments. (g) Effect of CD74 on MIF-induced transmigration of pro-myelocytic HL-60 cells. Promyelocytic HL-60 cells do not express measurable levels of surface CD74 as analyzed by flow cytometry (data not shown). HL-60 cells (2×10−6) were transiently transfected with the plasmid pcDNA3.11V5-HisTOPO-TA-CD74 (pCD74) or control vector (pcDNA3, 1 μg each) using Amaxa nucleofection technology (Cell Line Nucleofector Kit V) to ectopically express CD74, as confirmed by flow cytometry. Cells (5×104) labeled with calcein-AM for 2 h in RPMI 1640/10% FCS were placed in the upper chamber of 5 μm pore Transwell filters, and allowed to migrate towards MIF at indicated concentrations for 2 h. Migrated cells were quantitated by automated fluorescent analysis of calcein-positive cells using a Wallac Victor fluorescent plate reader at excitation/emission wavelengths of 485/535 nm. Data represent means±SD of 3-6 experiments and are expressed as chemotactic index.

FIG. 9. The carotid arteries of Apoe−/− mice, Mif+/+ Ldlr−/− and Mif−/− Ldlr−/− mice fed an atherogenic diet for 6 weeks or of Mif+/+ and Mif−/− mice after intraperitoneal pretreatment with Tnf-a for 4 h were perfused with calcein-labeled MonoMac6 cells, as indicated. MonoMac6 cells were pretreated with isotype control IgG, antibodies to CD74 (anti-CD74) or to CXCR2 (anti-CXCR2), as indicated. For Mif blockade, carotid arteries were preperfused with antibody to Mif (anti-Mif), and for additional loading, arteries were perfused with exogenous MIF (+MIF) for 2 h, as indicated. Shown are still frame images representative of at least three independent experiments depicting monocytes firmly adherent to the arterial wall and visualized by stroboscopic epifluorescence illumination.

FIG. 10 is an illustrative demonstration that the monoclonal Mif antibody NIHIII.D.9 specifically recognizes Mif, but not Cxcl1/Kc or CXCL8. (a) The Mif antibody NIHIII.D.9 specifically recognizes MIF but not Cxcl1/Kc as determined by ‘native’ immunoblotting assay (slot blot). 50 ng Cxcl1/Kc and 50 ng MIF were blotted on a nitrocellulose membrane under native buffer conditions using a slot blot apparatus as indicated (see arrows). Two other slots were left empty and treated with buffer alone for control purposes (control). The membrane was blocked and washed according to the manufacturer's recommendation (Amersham) and developed with a neutralizing antibody to MIF (NIHIII.D.9) comparable to a standard Western blotting protocol. After stripping, the membrane was re-probed with a rat anti-mouse Cxcl1/Kc antibody (MAB453) to verify Cxcl1/Kc presence (not shown). (b) The Mif antibody NIHIII.D.9 specifically recognizes MIF but not CXCL8 or CXCL12 as determined by ‘denaturating’ immunoblotting assay (Western blot). 50 ng CXCL8, CXCL12, and MIF were electrophoresed in a 4-12% denaturating and reducing NuPage gel, electro-blotted on a nitrocellulose membrane under denaturating transfer conditions following a routine Western blotting protocol (see Methods) and the blot developed with the neutralizing anti-Mif antibody NIHIII.D.9. The blot was stripped to verify the presence of the CXCL8 and CXCL12 bands (not shown). (c) The Mif antibody NIHIII.D.9 inhibits MIF-triggered but not CXCL8- or Cxcl1-triggered arrest of peripheral blood mononuclear cells (PBMCs). SVEC monolayers were preincubated with Cxcl1 (100 ng/mL) and either blocking antibodies to Cxcl1 (anti-Cxcl1) or Mif (anti-Mif) for 2 h and were perfused with murine PBMCs (1.5×105/mL in assay buffer) at a flow rate of 1.5 dyne/cm2 for 2 min (n=3). Cxcl1-triggered PBMC arrest was significantly inhibited by a blocking antibody to Cxcl1, whereas the Mif-blocking antibody NIHIII.D.9 did not alter Cxcl1-induced cell arrest. (d) Likewise, HUVEC monolayers were preincubated with CXCL8 (10 ng/mL), MIF (50 ng/mL) or the blocking antibody to MIF as indicated, and were perfused with human PBMCs (5×105/mL). The blocking Mif antibody significantly inhibited adhesion triggered by MIF but not by CXCL8 (n=3).

### DETAILED DESCRIPTION OF THE INVENTION

MIF has emerged as a key element in vascular processes giving rise to atherosclerosis. Its expression is upregulated in endothelial cells, smooth muscle cells (SMCs) and macrophages during the development of atherosclerotic lesions in humans, rabbits and mice. Genetic deletion of Mif retards diet-induced atherogenesis in LDL receptor-deficient (Ldlr^{-/-}), MIF-deficient mice. However, the pathway(s) by which MIF exerts its influence on atherosclerosis was not known.

As disclosed herein, MIF is a functional noncognate ligand for the chemokine receptors CXCR2 and CXCR4. As disclosed herein, MIF regulates leukocyte migration and activates inflammatory processes by activating at least one of its receptors CD74, CXCR2 or CXCR4. The present invention comprises methods of treating or preventing an inflammatory condition in a patient in need thereof comprising administering to said patient one or more antibodies that inhibit activation of CD74, CXCR2 or CXCR4 by MIF. Also, as disclosed herein, CD74 activates G-protein coupled receptors (GPCRs), activates CXCR2 and associates with these molecules into a signaling complex. Therefore the present invention also comprises methods of treating or preventing an inflammatory condition in a patient in need thereof comprising administering to said patient one or more antibodies that inhibit the activation GPCRs or CXCR2 by CD74. The present invention also comprises methods of treating or preventing atherosclerosis.

Exemplary Therapeutic Methods and Antibodies that Inhibit Activation of CD74, CXCR2 or CXCR4 Methods to Inhibit CXCR2 or CXCR4

The inhibition of CXCR2 or CXCR4 activity in accordance with the invention is accomplished in a number of ways and in some embodiments comprises the administration of one or more antibodies that inhibit the activation of CXCR2 or CXCR4. In some embodiments of the invention a single antibody is administered. In some embodiments, two antibodies are administered and in some embodiments three antibodies are administered. In some embodiments one antibody is administered that inhibits the activation of CXCR2 by MIF and another antibody is administered that inhibits the activation of CXCR4 by MIF.

### Targeting MIF

In some embodiments the present invention comprises a method of treating or preventing an inflammatory condition in a patient in need thereof comprising administering to said patient one or more antibodies that inhibit activation of CXCR2 or CXCR4 by MIF. In some embodiments the present invention comprises administering one or more antibodies that bind to MIF and inhibit its biological activity. In some embodiments the present invention comprises administering an antibody-derived antigen binding fragment that binds MIF. In some embodiments the antibody or antibody-derived binding fragment that binds MIF is or is derived from clone NIHIII.D.9 (Lan, H. Y. et al. (1997) J. Exp. Med. 185:1455-1465). In some embodiments the antibody or antibody-derived binding fragment that binds MIF is or is derived from clone IID.9, IIID.9, XIF7, I31, IV2.2, XI17, XII15.6 or XIV15.4 (U.S. Pat. No. 6,645,493). In some embodiments the present invention comprises administering an antibody or antibody-derived binding fragment that competes for the binding of clone NIHIII.D.9 to MIF. In some embodiments the present invention comprises administering an antibody or antibody-derived binding fragment that competes for the binding of clone IID.9, IIID.9, XIF7, I31, IV2.2, XI17, XII15.6, or XIV15.4 to MIF. In some embodiments the present invention comprises administering one or more antibodies that selectively inhibit the ability of MIF to activate CXCR2. In some embodiments the present invention comprises administering one or more antibodies that selectively inhibit the ability of MIF to activate CXCR4. In some embodiments the present invention comprises administering one or more antibodies that selectively inhibit the ability of MIF to activate CXCR2 and CXCR4. In some embodiments the present invention comprises administering one or more antibodies that selectively inhibit the ability of MIF to activate CXCR2, CXCR4 and CD74.

### Targeting the Pseudo-ELR Motif

Activation of CXCR2 by its cognate ligands requires an N-terminal Glu-Leu-Arg (ELR) motif. As disclosed herein, MIF features a pseudo-ELR motif, composed of two nonadjacent but adequately spaced residues (Asp and Arg) in exposed neighboring loops, that mimic the ELR motif found in chemokines. Therefore, in some embodiments, the present invention comprises a method of treating or preventing an inflammatory condition in a patient in need thereof comprising administering to said patient an antibody that inhibits activation of CXCR2 by MIF, wherein said antibody blocks binding of the pseudo-ELR motif to CXCR2. In some embodiments the invention comprises an antibody that that specifically binds the pseudo-ELR motif of MIF.

### Methods to Inhibit CXCR2

In some embodiments the present invention comprises a method of treating or preventing an inflammatory condition in a patient in need thereof comprising administering to said patient one or more antibodies that inhibit activation of CXCR2. In some embodiments the method comprises administering a CXCR2 antagonist. In some embodiments the method comprises administering one or more antibodies that bind CXCR2. In some embodiments the present invention comprises administering an antibody-derived antigen binding fragment that binds CXCR2. In some embodiments the antibody or antibody-derived antigen binding fragment is or is derived from Clone 48311.211 (Wells T N et. al. (1998) Pharmacol Sci 19:376-80). In some embodiments the present invention comprises administering an antibody or antibody-derived binding fragment that competes for the binding of clone 48311.211 to CXCR2. In some embodiments the method comprises administering one or more antibodies that bind CXCR2 and inhibit the ability of MIF to activate CXCR2. In some embodiments the method comprises administering one or more antibodies that selectively inhibits the ability of MIF to activate CXCR2, yet allows activation of CXCR2 by at least one other CXCR2 ligand. For example in some embodiments the anti-CXCR2 antibody that is administered selectively inhibits the ability of MIF to activate CXCR2, yet allows activation of CXCR2 by CXCL8. In some embodiments the method comprises administering a peptide mimetic or analog of MIF that binds CXCR2. In some embodiments the method comprises administering a peptide mimetic of MIF or analog of MIF that binds CXCR2 and inhibits activation of CXCR2 by endogenous MIF.

### Methods to Inhibit CXCR4

In some embodiments the present invention comprises a method of treating or preventing an inflammatory condition in a patient in need thereof comprising administering to said patient one or more antibodies that inhibit activation of CXCR4. In some embodiments the method comprises administering one or more antibodies that bind CXCR4. In some embodiments the present invention comprises administering an antibody-derived antigen binding fragment that binds CXCR4. In some embodiments the antibody or antibody-derived antigen binding fragment is or is derived from 44708 (R&D) or an antibody in the FABSP2 cocktail (R&D). In some embodiments the present invention comprises administering an antibody or antibody-derived binding fragment that competes for the binding of 44708 or an antibody in the FABSP2 cocktail. In some embodiments the method comprises administering one or more antibodies that bind CXCR4 and selectively inhibits the ability of MIF to activate CXCR4. In some embodiments the anti-CXCR4 antibody selectively inhibits the ability of MIF to activate CXCR4, yet allows activation of CXCR4 by at least one other CXCR4 ligand. For example in some embodiments the anti-CXCR4 antibody that is administered selectively inhibits the ability of MIF to activate CXCR4, yet allows activation of CXCR4 by CXCL12. In some embodiments the method comprises administering a peptide mimetic or analog of MIF that binds CXCR4. In some embodiments the peptide mimetic of MIF or analog of MIF that binds CXCR4 selectively inhibits activation of CXCR4 by endogenous MIF. In some embodiments the method comprises administering a small compound antagonist of CXCR4. In some embodiments the present invention comprises administering to said patient the CXCR4 antagonist AMD3465. In some embodiments the CXCR4 antagonist is selected from the list; COR100140 (a small molecule compound developed by Cortical Pty Ltd.), ALX40-4C (Doranz, B. J., et al. (2001) 17(6):475-486), AMD-070, AMD3100 (Stone N. D., et al. (2007) Antimicrob Agents Chemother. 51(7):2351-8), KRH-1636, KRH-2731 (Briz, V., et al. (2006) Journal of Antimicrobial Chemotherapy 57(4):619-627), KRH-3955, KRH-3140 (Tanaka, Y., et al. (2006) Conf Retrovir Opportunistic Infect, February 5-8; 13:abstract no. 49LB), AMD3465 (Hu, J. S., et al. (2006) Am J Pathol. 169(2): 424-432), T134, T22 (Arakaki, R., et al. (1999) Journal of Virology 73(2):1719-1723), T140, TC14012, TN14003 (Burger, M., et al. (2005) Blood 106(5):1824-1830), RCP168 (Zeng, Z., et al. (2006) Mol Cancer Ther. 5(12):3113-21), POL3026 (Moncunill G., et al., (2008) Mol Pharmacol, January 8; [Epub ahead of print]) and CTCE-0214 (Li, K., et al., (2006) Stem Cells 24(1) 55-64). In some embodiments the method comprises administering one or more small compounds that bind CXCR4 and selectively inhibits the ability of MIF to activate CXCR4. In some embodiments the CXCR4 binding small compound selectively inhibits the ability of MIF to activate CXCR4, yet allows activation of CXCR4 by at least one other CXCR4 ligand. For example the CXCR4 binding small compound that is administered can selectively inhibit the ability of MIF to activate CXCR4, yet allow activation of CXCR4 by CXCL12. In some embodiments the CXCR4-binding compound is an analog of MIF as described in U.S. Pat. No. 6,274,227 or U.S. Pat. No. 4,278,595. In some embodiments the small compound antagonist of MIF binds CXCR4 and selectively inhibits activation of CXCR4 by endogenous MIF.

### Methods to Inhibit Both CXCR2 and CXCR4

In some embodiments the present invention comprises a method of treating or preventing an inflammatory condition in a patient in need thereof comprising administering to said patient a composition of one or more antibodies that inhibit activation of CXCR2 and CXCR4. In some embodiments the method comprises administering one or more antibodies that bind CXCR4 or CXCR4. In some embodiments the method comprises administering an antibody-derived antigen binding fragment. In some embodiments the method comprises administering one or more antibodies that selectively inhibit the ability of MIF to activate CXCR2 and CXCR4. In some embodiments the method comprises administering a CXCR2/CXCR4-binding compound that is a peptide mimetic or analog of MIF. In some embodiments the peptide mimetic of MIF selectively inhibits activation of CXCR2 and CXCR4 by endogenous MIF. In some embodiments the method of treatment comprises administering a MIF antagonist.

### Methods to Inhibit CXCR4 on T-Cells

As disclosed herein MIF binds T-cells and activates CXCR4. Therefore the present invention comprises a method of treating or preventing atherosclerosis in a patient in need thereof comprising administering to said patient one or more antibodies that selectively inhibit MIF-dependent activation of CXCR4 on T-cells.

### Methods to Inhibit CD74

The present invention comprises a method of treating or preventing atherosclerosis in a patient in need thereof comprising administering to said patient one or more antibodies that inhibit activation of CD74 by MIF. In some embodiments the method comprises administering an anti-CD74 antibody. In some embodiments the anti-CD74 antibody is or is derived from M-B741 (Pharmingen).

As disclosed herein, CD74 activates G-protein coupled receptors, forms complexes with CXCR2 and induces inflammatory processes by MY-dependent or MIF-independent mechanisms. Therefore the present invention comprises methods of inhibiting CD74-mediated activation of a G-protein coupled receptor or CD74-mediated activation of CXCR2. In some embodiments the present invention comprises a method of treating or preventing atherosclerosis in a patient in need thereof comprising administering to said patient one or more antibodies that inhibit CD74-mediated activation of a G-protein coupled receptor. In some embodiments the present invention comprises a method of treating or preventing atherosclerosis in a patient in need thereof comprising administering to said patient one or more antibodies that inhibit CD74 binding to CXCR2. In some embodiments the present invention comprises administering a antibody that interferes with the ability of CD74 to form a complex with CXCR2. In some embodiments the present invention comprises a method of treating or preventing atherosclerosis in a patient in need thereof comprising administering to said patient one or more antibodies that inhibit MIF-independent activation of a GPCR by CD74.

### Atherosclerosis

In some embodiments, the methods described herein are used to treat a subject suffering from atherosclerosis or a condition that is associated with atherosclerosis. In some embodiments the methods provided herein are used at any stage of atherosclerotic plaque development. According to a new classification adopted by the American Heart Association, eight lesion types are presented during the progression of atherosclerosis.

Type I lesions are formed by small lipid deposits (intracellular and in macrophage foam cells) in the intima and cause the initial and most minimal changes in the arterial wall. Such changes do not thicken the arterial wall.

Type II lesions are characterized by fatty streaks including yellow-colored streaks or patches that increase the thickness of the intima by less than a millimeter. They consist of accumulation of more lipid than is observed in type I lesions. The lipid content is approximately 20-25% of the dry weight of the lesion. Most of the lipid is intracellular, mainly in macrophage foam cells, and smooth muscle cells. In some embodiments of Type II lesions the extracellular space contains lipid droplets, but these are smaller than those within the cell, and small vesicular particles. These lipid droplets have previously been described as consisting of cholesterol esters (cholesteryl oleate and cholesteryl linoleate), cholesterol, and phospholipids.

Type III lesions are also described as preatheroma lesions. In type III lesions the intima is thickened only slightly more than observed for type II lesions. Type III lesions do not obstruct arterial blood flow. The extracellular lipid and vesicular particles are identical to those found in type II lesions, but are present in increased amount (approx. 25-35% dry weight) and start to accumulate in small pools.

Type IV lesions are associated with atheroma. They are crescent-shaped and increase the thickness of the artery. In some embodiments the lesion does not narrow the arterial lumen much except for persons with very high plasma cholesterol levels. In some embodiments and for many people, the type IV lesion is not visible by angiography. Type IV lesions consist of an extensive accumulation (approx. 60% dry weight) of extracellular lipid in the intimal layer (sometimes called a lipid core). In some embodiments the lipid core contains small clumps of minerals. These lesions are susceptible to rupture and to formation of mural thrombi.

Type V lesions are associated with fibroatheroma. They have one or multiple layers of fibrous tissue consisting mainly of type I collagen. Type V lesions have increased wall thickness and, as the atherosclerosis progresses increased reduction of the lumen. These lesions have features that permit further subdivision. In type Va lesions, new tissue is part of a lesion with a lipid core. In type Vb lesions, the lipid core and other parts of the lesion are calcified (leading to Type VII lesions). In type Vc lesions, the lipid core is absent and lipid generally is minimal (leading to Type VIII lesions). Generally, the lesions that undergo disruption are type Va lesions. They are relatively soft and have a high concentration of cholesterol esters rather than free cholesterol monohydrate crystals. In some cases Type V lesions rupture and form mural thrombi.

Type VI lesions are complicated lesions having disruptions of the lesion surface such as fissures, erosions or ulcerations (Type VIa), hematoma or hemorrhage (Type VIb), and thrombotic deposits (Type VIc) that are superimposed on Type IV and V lesions. Type VI lesions have increased lesion thickness and the lumen is often completely blocked. In some cases these lesions convert to type V lesions, but they are larger and more obstructive.

Type VII lesions are calcified lesions characterized by large mineralization of the more advanced lesions. Mineralization takes the form of calcium phosphate and apatite, replacing the accumulated remnants of dead cells and extracellular lipid.

Type VIII lesions are fibrotic lesions consisting mainly of layers of collagen, with little lipid. In some embodiments Type VIII lesions are a consequence of lipid regression of a thrombus or of a lipidic lesion with an extension converted to collagen. In some embodiments these lesions obstruct the lumen of medium-sized arteries.

In some embodiments, the methods described herein are used to treat a subject suffering from atherosclerosis (or arteriosclerosis), a subject predisposed to atherosclerosis, or a subject suffering from a condition that is associated with atherosclerosis. Examples of conditions that are treated or prevented with the methods of the invention include but are not limited to atherosclerosis (or arteriosclerosis), preeclampsia, peripheral vascular disease, peripheral artery occlusive disease, heart disease (cardiovascular disease), congenital heart disease, stroke, angina, acute coronary syndromes including unstable angina, thrombosis and myocardial infarction, plaque rupture, stenosis, both primary and secondary (in-stent) restenosis in coronary or peripheral arteries, transplantation-induced sclerosis, peripheral limb disease, intermittent claudication and diabetic complications (including ischemic heart disease, peripheral artery disease, congestive heart failure, retinopathy, neuropathy and nephropathy), thrombosis, hypertension, pulmonary hypertension, aneurysms, infarction, myocardial infarction, cerebral ischemia, and cardiac ischemia.

### Atherosclerotic Plaque

In some embodiments the present invention comprises a method of inducing regression of pre-existing atherosclerotic plaques in a patient in need thereof comprising administering to the patient one or more agents that inhibit activation of CXCR2 and CXCR4 by MIF and/or binding of MIF to CXCR2 and CXCR4. In some embodiments the present invention comprises a method of stabilizing atherosclerotic plaques in a patient in need thereof comprising administering to the patient one or more agents that inhibit activation of CXCR2 and CXCR4 by MIF and/or binding of MIF to CXCR2 and CXCR4. In some embodiments the present invention comprises inducing a more stable plaque phenotype.

### Cell Recruitment

The migration of leukocytes to areas of inflammation (e.g. atherosclerotic lesions) and into the surrounding tissues is referred to as inflammatory cell recruitment. In some embodiments the present invention comprises a method of inhibiting inflammatory cell recruitment to atherosclerotic lesions in a patient in need thereof comprising administering to the patient one or more agents that inhibit activation of CXCR2 and CXCR4 by MIF and/or binding of MIF to CXCR2 and CXCR4. The migration of leukocytes to atherosclerotic lesions and into the surrounding tissues is referred to as atherogenic cell recruitment. In some embodiments the present invention comprises a method inhibiting atherogenic cell recruitment in a patient in need thereof comprising administering to the patient one or more agents that inhibit activation of CXCR2 and CXCR4 by MIF and/or binding of MIF to CXCR2 and CXCR4. In some embodiments the present invention comprises a method of reducing macrophage content and T-cell content of an atherosclerotic plaque or atherosclerotic lesion.

### Autoimmune Disorders

In some embodiments, the methods described herein are used to treat a patient in need thereof suffering from an autoimmune disorder. Examples of autoimmune disorders include, but are not limited to colitis, multiple sclerosis, arthritis, rheumatoid arthritis, osteoarthritis, juvenile arthritis, psoriatic arthritis, acute pancreatitis, chronic pancreatitis, atherosclerosis, inflammatory bowel disease, Crohn's disease, ulcerative colitis, multiple sclerosis, autoimmune hemolytic syndromes, autoimmune hepatitis, autoimmune neuropathy, autoimmune ovarian failure, autoimmune orchitis, autoimmune thrombocytopenia, reactive arthritis, diabetes, ankylosing spondylitis, silicone implant associated autoimmune disease, Sjogren's syndrome, systemic lupus erythematosus, vasculitis syndromes (such as, for example, giant cell arteritis, Behcet's disease & Wegener's granulomatosis), Vitiligo, secondary hematologic manifestation of autoimmune diseases (such as, for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (including, for example, Meniere's disease), Goodpasture's syndrome, Graves' disease, HIV-related autoimmune syndromes and Gullain-Barre disease.

### Inflammatory Conditions

In some embodiments, the methods described herein are used to treat a patient in need thereof suffering from an inflammatory condition. Examples of inflammatory conditions include, but are not limited to sepsis, septic shock, endotoxic shock, exotoxin-induced toxic, gram negative sepsis, toxic shock syndrome, glomerulonephritis, peritonitis, interstitial cystitis, psoriasis, atopic dermatitis, hyperoxia-induced inflammations, chronic obstructive pulmonary disease (COPD), vasculitis, graft vs. host reaction (i.e., graft vs. host disease), allograft rejections (e.g., acute allograft rejection, and chronic allograft rejection), early transplantation rejection (e.g., acute allograft rejection), reperfusion injury, pancreatitis, chronic infections, meningitis, encephalitis, myocarditis, gingivitis, post surgical trauma, tissue injury, traumatic brain injury, hepatitis, enterocolitis, sinusitis, uveitis, ocular inflammation, optic neuritis, scleritis, polymyositis, gastric ulcers, esophagitis, peritonitis, periodontitis, dermatomyositis, gastritis, myositis, polymyalgia, pneumonia and bronchitis.

### Examples of Pharmaceutical Compositions and Methods of Administration

Pharmaceutical compositions are formulated using one or more physiologically acceptable carriers including excipients and auxiliaries which facilitate processing of the active antibodies into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include a MIF receptor inhibitor and a pharmaceutically acceptable diluent(s), excipient(s), or carrier(s). In addition, MIF receptor inhibitors are optionally administered as pharmaceutical compositions in which they are mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a MIF receptor inhibitor with other chemical components, such as carriers, stabilizers, diluents, dispersing agents, suspending agents, thickening agents, and/or excipients. The pharmaceutical composition facilitates administration of MIF receptor inhibitor to an organism. In practicing the methods of treatment or use provided herein, therapeutically effective amounts of A MIF receptor inhibitor are administered in a pharmaceutical composition to a mammal having a condition, disease, or disorder to be treated. Preferably, the mammal is a human. A therapeutically effective amount varies depending on the severity and stage of the condition, the age and relative health of the subject, the potency of the MIF receptor inhibitor used and other factors. MIF receptor inhibitors are optionally used singly or in combination with one or more therapeutic agents as components of mixtures.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions will include a MIF receptor inhibitor, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of MIF receptor inhibitors having the same type of activity. In some situations, MIF receptor inhibitors exist as tautomers. All tautomers are included within the scope of the compounds presented herein. Additionally, in some embodiments, a MIF receptor inhibitor exists in unsolvated as well as solvated forms with pharmaceutically acceptable solvents such as water, ethanol, and the like. The solvated forms of the MIF receptor inhibitors presented herein are also considered to be disclosed herein.

“Carrier materials” include any commonly used excipients in pharmaceutics and should be selected on the basis of compatibility with antibodies disclosed herein, such as a MIF receptor inhibitor, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include a MIF receptor inhibitor, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use are optionally obtained by mixing one or more solid excipients with a MIF receptor inhibitor, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active antibody doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of MIF receptor inhibitor are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a MIF receptor inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which includes a MIF receptor inhibitor, are optionally further formulated to provide a controlled release of a MIF receptor inhibitor. Controlled release refers to the release of a MIF receptor inhibitor from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include a MIF receptor inhibitor, are delivered using a pulsatile dosage form. A pulsatile dosage form is capable of providing one or more immediate release pulses at predetermined time points after a controlled lag time or at specific sites. Pulsatile dosage forms including the formulations described herein, which include a MIF receptor inhibitor, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a MIF receptor inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are elf-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, a MIF receptor inhibitors is optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of a MIF receptor inhibitor and a suitable powder base such as lactose or starch.

Buccal formulations that include a MIF receptor inhibitor include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of a MIF receptor inhibitor, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co-polymers, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of a MIF receptor inhibitor is administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) a formulation of at least one antibody that inhibits activation of CD74, CXCR2 or CXCR4; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of a MIF receptor inhibitor employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of a MIF receptor inhibitor is optionally accomplished by means of iontophoretic patches and the like. Additionally, transdermal patches provide controlled delivery of a MIF receptor inhibitor. The rate of absorption is optionally slowed by using rate-controlling membranes or by trapping a MIF receptor inhibitor within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing a MIF receptor inhibitor optionally with carriers, optionally a rate controlling barrier to deliver a MIF receptor inhibitor to the skin of the host at a controlled and predetermined rate over a prolonged period of time, and means to secure the device to the skin.

Formulations that include a MIF receptor inhibitor suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a MIF receptor inhibitor is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of a MIF receptor inhibitor in water soluble form. Additionally, suspensions of a MIF receptor inhibitor are optionally prepared as appropriate oily injection suspensions.

In some embodiments, a MIF receptor inhibitor is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

A MIF receptor inhibitor is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

A MIF receptor inhibitor is optionally used in the preparation of medicaments for the prophylactic and/or therapeutic treatment of inflammatory conditions or conditions that would benefit, at least in part, from amelioration. In addition, a method for treating any of the diseases or conditions described herein in a subject in need of such treatment, involves administration of pharmaceutical compositions containing a MIF receptor inhibitor as described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In the case wherein the patient's condition does not improve, upon the doctor's discretion the administration of a MIF receptor inhibitor is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of a MIF receptor inhibitor is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical composition described herein are in unit dosage forms suitable for single administration of precise dosages. In unit dosage form, the formulation is divided into unit doses containing appropriate quantities of a MIF receptor inhibitor. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for a MIF receptor inhibitor are from about 0.01 to 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the MIF receptor inhibitor used, the disease or condition to be treated, the mode of administration, the requirements of the individual subject, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. A MIF receptor inhibitor exhibiting high therapeutic indices are preferred. The data obtained from cell culture assays and animal studies is optionally used in formulating a range of dosage for use in human. The dosage of such a MIF receptor inhibitor lies preferably within a range of circulating concentrations that include the ED50 with minimal toxicity. The dosage optionally varies within this range depending upon the dosage form employed and the route of administration utilized.

### Combination Treatments

MIF receptor inhibitor compositions described herein are also optionally used in combination with other therapeutic reagents that are selected for their therapeutic value for the condition to be treated. In general, the compositions described herein and, in embodiments where combinational therapy is employed, other agents do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

In certain instances, it is appropriate to administer a MIF receptor inhibitor composition as described herein in combination with another therapeutic agent. By way of example only, if one of the side effects experienced by a patient upon receiving a MIF receptor inhibitor composition as described herein is nausea, then it is appropriate to administer an anti-nausea agent in combination with the initial therapeutic agent. Or, by way of example only, the therapeutic effectiveness of a MIF receptor inhibitor are enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering a MIF receptor inhibitor with another therapeutic agent (which also includes a therapeutic regimen) that also has therapeutic benefit. In any case, regardless of the disease, disorder or condition being treated, the overall benefit experienced by the patient is either simply additive of the two therapeutic agents or the patient experiences a synergistic benefit.

Therapeutically-effective dosages vary when the drugs are used in treatment combinations. Methods for experimentally determining therapeutically-effective dosages of drugs and other agents for use in combination treatment regimens are documented methodologies. One example of such a method is the use of metronomic dosing, i.e., providing more frequent, lower doses in order to minimize toxic side effects. Combination treatment further includes periodic treatments that start and stop at various times to assist with the clinical management of the patient.

In any case, the multiple therapeutic agents (one of which is a MIF receptor inhibitor as described herein) are administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also be administered sequentially, with either therapeutic antibody being administered by a regimen calling for two-step administration. The two-step administration regimen optionally calls for sequential administration of the active agents or spaced-apart administration of the separate active agents. The time period between the multiple administration steps ranges from, a few minutes to several hours, depending upon the properties of each pharmaceutical agent, such as potency, solubility, bioavailability, plasma half-life and kinetic profile of the pharmaceutical agent. Circadian variation of the target molecule concentration are optionally used to determine the optimal dose interval.

In addition, a MIF receptor inhibitor is optionally used in combination with procedures that provide additional or synergistic benefit to the patient. By way of example only, patients are expected to find therapeutic and/or prophylactic benefit in the methods described herein, wherein pharmaceutical compositions of a MIF receptor inhibitor and/or combinations with other therapeutics are combined with genetic testing to determine whether that individual is a carrier of a mutant gene that is correlated with certain diseases or conditions.

A MIF receptor inhibitor and the additional therapy(ies) are optionally administered before, during or after the occurrence of a disease or condition, and the timing of administering the composition containing a MIF receptor inhibitor varies in some embodiments. Thus, for example, a MIF receptor inhibitor is used as a prophylactic and is administered continuously to subjects with a propensity to develop conditions or diseases in order to prevent the occurrence of the disease or condition. A MIF receptor inhibitor and compositions are optionally administered to a subject during or as soon as possible after the onset of the symptoms. The administration of the antibodies are optionally initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration is optionally via any route practical, such as, for example, an intravenous injection, a bolus injection, infusion over 5 minutes to about 5 hours, a pill, a capsule, transdermal patch, buccal delivery, and the like, or combination thereof. A MIF receptor inhibitor is preferably administered as soon as is practicable after the onset of a disease or condition is detected or suspected, and for a length of time necessary for the treatment of the disease, such as, for example, from about 1 month to about 3 months. The length of treatment optionally varies for each subject, and the length is then determined using the known criteria. For example, a MIF receptor inhibitor or a formulation containing a MIF receptor inhibitor are administered for at least 2 weeks, preferably about 1 month to about 5 years, and more preferably from about 1 month to about 3 years.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that in some embodiments of the invention various alternatives to the embodiments described herein are employed in practicing the invention.

Exemplary Therapeutic Agents for Use in Combination with Antibodies that Inhibit Activation of CD74, CXCR2 or CXCR4

### Agents for Treating Atherosclerosis

Where a subject is suffering from or at risk of suffering from atherosclerosis or a condition that is associated with atherosclerosis, a MIF receptor inhibitor composition described herein is optionally used together with one or more agents or methods for treating atherosclerosis or a condition that is associated with atherosclerosis in any combination. Examples of therapeutic agents/treatments for treating atherosclerosis or a condition that is associated with atherosclerosis include, but are not limited to any of the following: torcetrapib, aspirin, niacin, HMG CoA reductase inhibitors (e.g., atorvastatin, fluvastatin, lovastatin, pravastatin, rosuvastatin and simvastatin), colesevelam, cholestyramine, colestipol, gemfibrozil, probucol and clofibrate.

### Agents for Treating Autoimmune Disorders

Where a subject is suffering from or at risk of suffering from an autoimmune disorder, a MIF receptor inhibitor composition described herein is optionally used together with one or more agents or methods for treating autoimmune disorder in any combination. Examples of therapeutic agents/treatments for treating autoimmune disorders include, but are not limited to any of the following:

### Agents for Treating Inflammation

Where a subject is suffering from or at risk of suffering from an inflammatory condition, a MIF receptor inhibitor composition described herein is optionally used together with one or more agents or methods for treating an inflammatory condition in any combination. Examples of therapeutic agents/treatments for treating an inflammatory condition include, but are not limited to any of the following: corticosteroids, nonsteroidal anti-inflammatory drugs (NSAID) (e.g. ibuprofen, naproxen, acetominophen, aspirin, Fenoprofen (Nalfon), Flurbiprofen (Ansaid), Ketoprofen, Oxaprozin (Daypro), Diclofenac sodium (Voltaren), Diclofenac potassium (Cataflam), Etodolac (Lodine), Indomethacin (Indocin), Ketorolac (Toradol), Sulindac (Clinoril), Tolmetin (Tolectin), Meclofenamate (Meclomen), Mefenamic acid (Ponstel), Nabumetone (Relafen), Piroxicam (Feldene), cox-2 inhibitors (e.g. celecoxib (Celebrex))), immunosuppressants (e.g. methotrexate (Rheumatrex), leflunomide (Arava), azathioprine (Imuran), cyclosporine (Neoral, Sandimmune) and cyclophosphamide (Cytoxan), Tumor Necrosis Factor (TNF) blockers (e.g. etanercept (Enbrel), infliximab (Remicade) and adalimumab (Humira)), Abatacept (CTLA4-Ig) and interleukin-1 receptor antagonists (e.g. Anakinra (Kineret).

In some embodiments any of the foregoing are utilized individually or in combination to inhibit the activation of any desired combination of CXCR2, CXCR4 & CD74 for the treatment of the relevant conditions, and further, are combined with any other anti-cytokine therapy (including steroid therapy), anti-initiator therapy, inhibitory cytokines or any combination thereof.

### EXAMPLES

The following specific examples are to be construed as merely illustrative, and not limitative of the remainder of the disclosure in any way whatsoever.

### Example 1

### Cell Lines and Reagents

Human aortic (Schober, A., et al. (2004) Circulation 109, 380-385) and umbilical vein (Weber, K. S., et al. (1999) Eur. J. Immunol. 29, 700-712) endothelial cells (PromoCell), MonoMac6 cells (Weber, C., et al. (1993) Eur. J. Immunol. 23, 852-859) and Chinese hamster ovary (CHO) ICAM-1-transfectants (Osterman, G., et al. (2002) Nat. Immunol. 3, 151-158) were used as described. Jurkat cells and RAW264.7 macrophages were transfected with pcDNA3-CXCR2. HL-60 cells were transfected with pcDNA3.1/V5-HisTOPO-TA-CD74 or vector control (Nucleofector Kit V, Amaxa). L1.2 cells were transfected with pcDNA3-CXCRs or pcDNA-CCR5 (UMR cDNA Resource Center) for assays on simian virus-40-transformed mouse microvascular endothelial cells (SVECs). Peripheral blood mononuclear cells were prepared from buffy coats, monocytes by adherence or immunomagnetic separation (Miltenyi), primary T cells by phytohaemaglutinin/interleukin-2 (Biosource) stimulation and/or immunomagnetic selection (antibody to CD3/M-450 Dynabeads), and neutrophils by Ficoll gradient centrifugation. Human embryonal kidney-CXCR2 transfectants (HEK293-CXCR2) have been described previously (Ben-Baruch, A., et al. (1997) Cytokine 9, 37-45).

Recombinant MIF was expressed and purified as described (Bemhagen, J., et al. (1993) Nature 365, 756-759). Chemokines were from PeproTech. Human VCAM-1.Fc chimera, blocking antibodies to CXCR1 (42705, 5A12), CXCR2 (48311), CXCR4 (44708, FABSP2 cocktail, R&D), human MIF and mouse MIF (NIHIII.D.9) (Lan, H. Y., et al. (1997) J. Exp. Med. 185, 1455-1465), CD74 (M-B741, Pharmingen), β₂ integrin (TS1/18), α₄ integrin (HP2/1) (Weber, C., et al. (1996) J. Cell Biol. 134, 1063-1073) and CXCR2 (RII115), and antibody to α_{L} integrin (327C) (Shamri, R., et al. (2005) Nat. Immunol. 6, 497-506) were used. PTX and B-oligomer were from Merck.

### Methods Used in Examples

Adhesion assays. Arrest of calcein-AM (Molecular Probes)-labeled monocytes, T cells and L1.2 transfectants was quantified in parallel-wall chambers in flow (1.5 dynes/cm², 5 min) (Schober, A., et al. (2004) Circulation 109, 380-385; Osterman, G., et al. (2002) Nat. Immunol. 3, 151-158; Weber, C., et al. (1996) J. Cell Biol. 134, 1063-1073). Confluent endothelial cells, CHO-ICAM-1 cells, VCAM-1.Fc-coated plates and leukocytes were pretreated with MIF, chemokines or antibodies. CHO-ICAM-1 cells incubated with MIF (2 h) were stained with antibody to MIF Ka565 (Leng, L., et al. (2003) J. Exp. Med. 197, 1467-1476) and FITC-conjugated antibody.

Chemotaxis assays. Using Transwell chambers (Costar), we quantified primary leukocyte migration toward MIF or chemokines by fluorescence microscopy or using calcein-AM labeling and FluoroBlok filters (Falcon). Cells were pretreated with PTX/B-oligomer, Ly294002, MIF (for desensitization), antibodies to CXCRs or CD74, or isotype IgG. Pore sizes and intervals were 5 μm and 3 h (monocytes), 3 μm and 1.5 h (T cells), and 3 mm and 1 h (neutrophils).

Q-PCR and ELISA. RNA was reverse-transcribed using oligo-dT primers. RTPCR was performed using QuantiTect Kit with SYBRGreen (Qiagen), specific primers and an MJ Opticon2 (Biozym). CXCL8 was quantified by Quantikine ELISA (R&D).

α_{L}β₂ integrin activation assay. Monocytes stimulated with MIF or Mg²⁺/EGTA (positive control) were fixed, reacted with the antibody 327C and an FITC-conjugated antibody to mouse IgG. LFA-1 activation analyzed by flow cytometry is reported as the increase in mean fluorescent intensity (MFI) or relative to the positive control (Shamri, R., et al. (2005) Nat. Immunol. 6, 497-506).

Calcium mobilization. Neutrophils or L1.2 CXCR2 transfectants were labeled with Fluo-4 AM (Molecular Probes). After the addition of the first or a subsequent stimulus (MIF, CXCL8 or CXCL7), MFI was monitored as a measure of cytosolic Ca²⁺ concentrations for 120 s using a BD FACSAria. L1.2 controls showed negligible calcium influx.

Receptor-binding assays. Because iodinated MIF is inactive (Leng, L., et al. (2003) J. Exp. Med. 197, 1467-1476; Kleemann, R., et al. (2002) J. Interferon Cytokine Res. 22, 351-363), competitive receptor binding (Hayashi, S., et al. (1995) J. Immunol. 154, 814-824) were performed using radioiodinated tracers (Amersham): [I¹²⁵]CXCL8, reconstituted at 4 nM (80 μCi/ml) to a final concentration of 40 pM; [I¹²⁵]CXCL12, reconstituted at 5 nM (100 μCi/ml) to a final concentration of 50 pM. For competition of [I¹²⁵]CXCL8 with MIF for CXCR2 binding or competition of [I¹²⁵]CXCL12 with MIF for CXCR4 binding in equilibrium binding assays, cold MIF and/or CXCL with tracers to HEK293-CXCR2 or CXCR4-bearing Jurkat cells were added. The analysis was performed by liquid scintillation counting. To calculate EC⁵⁰ and K_{d} values, a one-site receptor-ligand binding model was assumed and the Cheng/Prusoff-equation and GraphPad Prism were used.

For pull-down of biotin-MIF-CXCR complexes, HEK293-CXCR2 transfectants or controls were incubated with biotin-labeled MIF (Kleemann, R., et al. (2002) J. Interferon Cytokine Res. 22, 351-363), washed and lysed with coimmunoprecipitation (CoIP) buffer. Complexes were isolated from cleared lysates by streptavidin-coated magnetic beads (M280, Dynal) and analyzed by western blotting with antibody to CXCR2 or streptavidin-peroxidase. For flow cytometry, HEK293-CXCR2 transfectants or Jurkat cells pretreated with AMD3465 and/or a 20-fold excess of unlabeled MIF were incubated with fluorescein-labeled MIF and analyzed using a BD FACSCalibur.

CXCR internalization assays. HEK293-CXCR2 or Jurkat cells were treated with CXCL8 or CXCL12, respectively, treated with MIF, washed with acidic glycine-buffer, stained with antibodies to CXCR2 or CXCR4, and analyzed by flow cytometry. Internalization was calculated relative to surface expression of buffer-treated cells (100% control) and isotype control staining (0% control): geometric MFI[experimental]−MFI[0% control]/MFI[100% control]−MFI[0% control]×100.

Co localization of CXCR2 and CD74. RAW264.7-CXCR2 transfectants were co stained with CXCR2 and rat antibody to mouse CD74 (In-1, Pharmingen), followed by FITC-conjugated antibody to rat IgG and Cy3-conjugated antibody to mouse IgG, and were analyzed by confocal laser scanning microscopy (Zeiss).

Coimmunoprecipitation of CXCR2 and CD74. HEK293-CXCR2 cells transiently transfected with pcDNA3.1/V5-HisTOPO-TA-CD74 were lysed in nondenaturing CoIP buffer. Supernatants were incubated with the CXCR2 antibody RII115 or an isotype control, and were preblocked with protein G-sepharose overnight. Proteins were analyzed by western blots using an antibody to the His-tag (Santa Cruz). Similarly, CoIPs and immunoblots were performed with antibodies to the His-tag and CXCR2, respectively. L1.2-CXCR2 cells were subjected to immunoprecipitation with antibody to CXCR2 and immunoblotting with an antibody to mouse CD 74.

Ex vivo perfusion and intravital microscopy of carotid arteries. Mif^{-/-} Ldlr^{-/-} mice and Mif^{+/+} Ldlr^{-/-} littermate controls, crossbred from Mif^{-/-} (Fingerle-Rowson, G., et al. (2003) Proc. Natl. Acad. Sci. USA 100, 9354-9359) and Ldlr^{-/-} mice (Charles River), and Apoe^{-/-} mice were fed an atherogenic diet (21% fat; Altromin) for 6 weeks. All single knockout strains had been back-crossed in the C57BL/6 background ten times. Mit^{+/+} and Mif^{-/-} mice were treated with TNF-α (intraperitoneally (i.p.), 4 h). Explanted arteries were transferred onto the stage of an epifluorescence microscope and perfused at 4 μl/min with calcein-AM-labeled MonoMac6 cells treated with antibodies to CD74 or CXCR2, isotype control IgG, or left untreated (Huo, Y., et al. (2001) J. Clin. Invest. 108, 1307-1314). Untreated monocytic cells were perfused after blockade with antibody to MIF for 30 min. For intravital microscopy, rhodamine-G (Molecular Probes) was administered intravenously (i.v.), and carotid arteries were exposed in anesthetized mice. Arrest (>30 s) of labeled leukocytes was analyzed by epifluorescence microscopy (Zeiss Axiotech, 20× water immersion). All studies were approved by local authorities (Bezirksregierung Köln), and complied with German animal protection law Az: 50.203.2-AC 36, 19/05.

Mouse model of atherosclerotic disease progression. Apoe^{-/-} mice fed an atherogenic diet for 12 weeks were injected (3 injections per week, each 50 μg) with antibodies to MIF (NIHIIID.9), CXCL12 (79014) or CXCL1 (124014, R&D) (n=6-10 mice) for an additional 4 weeks. Aortic roots were fixed by in situ perfusion and atherosclerosis was quantified by staining transversal sections with Oil-Red-O. Relative macrophage and T-cell contents were determined by staining with antibodies to MOMA-2 (MCA519, Serotec) or to CD3 (PC3/188A, Dako) and FITC-conjugated antibody. In Mif^{-/-} Ldl^{r-/-} and Mif^{+/+} Ldlr^{-/-} mice fed a chow diet for 30 weeks, the abundance of luminal monocytes and lesional macrophages in aortic roots was determined as described (Verschuren, L., et al. (2005) Arterioscler. Thromb. Vase. Biol. 25, 161-167).

Cremaster microcirculation model. Human MIF (1 μg) was injected intra-scrotally and the cremaster muscle was exteriorized in mice treated with antibody to CXCR2 (100 μg i.p.). After 4 h, intravital microscopy (Zeiss Axioplan; 20×) was performed in postcapillary venules (Gregory, J. L., et al. (2004) Arthritis Rheum. 50, 3023-3034; Keane, M. P., et al. (2004) J. Immunol. 172, 2853-2860). Adhesion was measured as leukocytes stationary for more than 30 s, emigration as the number of extravascular leukocytes per field.

Bone marrow transplantation. Femurs and tibias were aseptically removed from donor Il8rb^{-/-} (Jackson Laboratories) or BALB/c mice. The cells, flushed from the marrow cavities, were administered i.v. into Mif^{+/+} or Mif^{-/-} mice 24 h after ablative whole-body irradiation (Zernecke, A., et al. (2005) Circ. Res. 96, 784-791).

Model of acute peritonitis. Mice repopulated with Il8rb^{+/+} or Il8rb^{-/-} bone marrow were injected i.p. with MIF (200 ng). After 4 h, peritoneal Iavage was performed and Gr-1⁺CD115⁻F4/80⁻ neutrophils were quantified by flow cytometry using the relevant conjugated antibodies.

Statistical analysis. Statistical analysis was performed using either a one-way analysis of variance (ANOVA) and Newman-Keuls post-hoc test or an unpaired Student's t-test with Welch's correction (GraphPad Prism).

### Example 2

### Surface-Bound MIF Induced Monocyte Arrest Through CXCR2

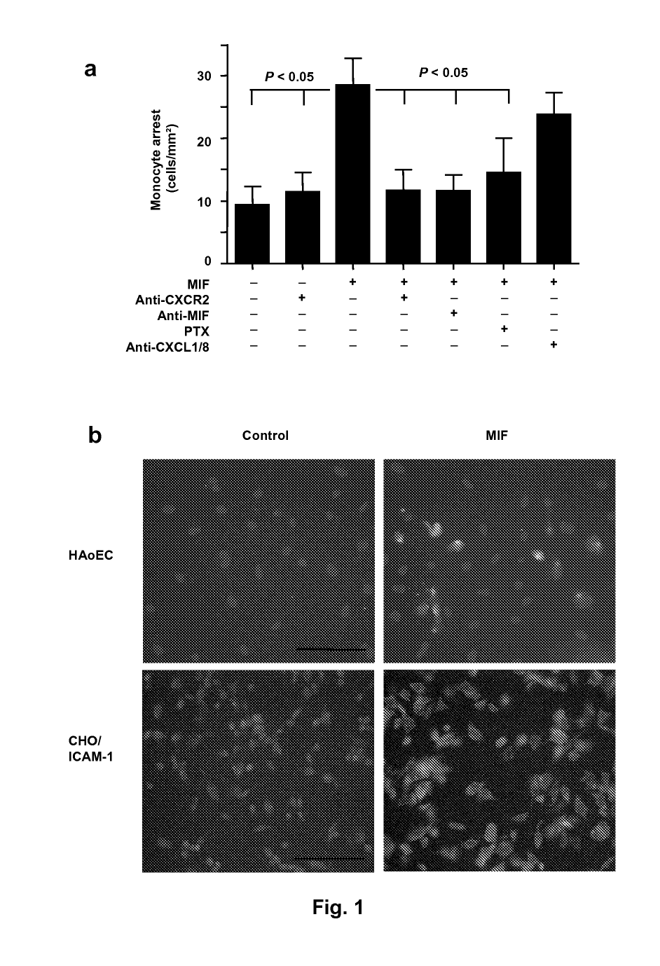

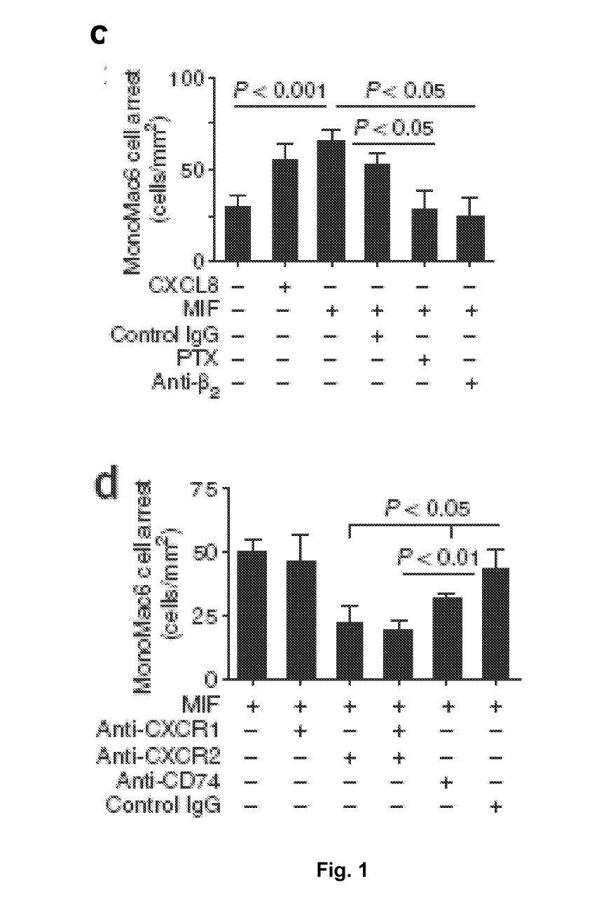

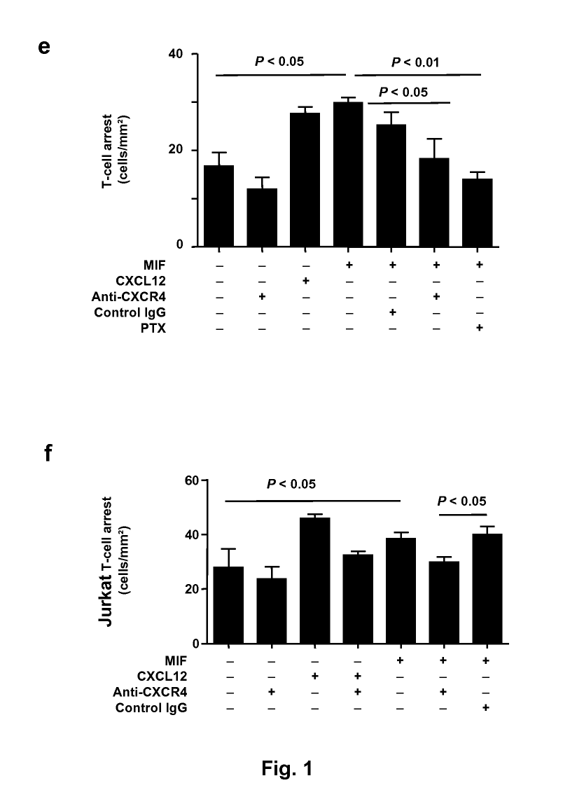

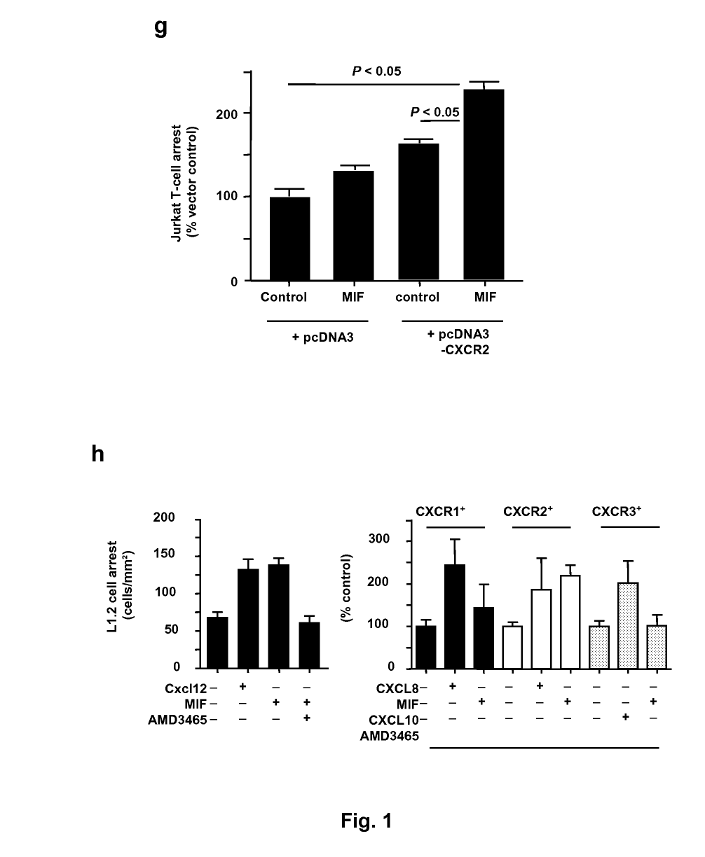

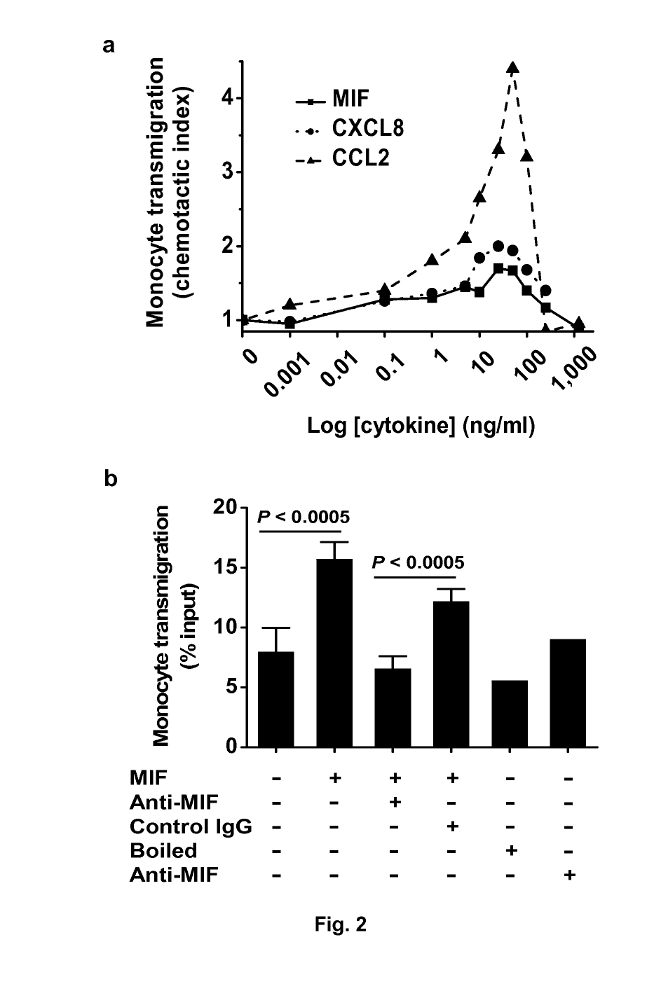

Monoclonal antibodies and pertussis toxin (PTX) were used to explore whether MIF-induced monocyte arrest depends on G_{αi}-coupled activities of CXCR2. Human aortic endothelial cells that had been pretreated with recombinant MIF for 2 h substantially increased the arrest of primary human monocytes under flow conditions, an effect blocked by an antibody to MIF (FIG. 1a). Notably, MIF-triggered, but not spontaneous, monocyte arrest was ablated by an antibody to CXCR2 or by PTX, implicating G_{αi}-coupled CXCR2. The ability of MIF to induce monocyte arrest through CXCR2 was confirmed using monocytic Mono-Mac6 cells and this activity was associated with an immobilization of MIF on aortic endothelial cells (FIG. 1b). This data indicated that MIF was presented on the endothelial cell surface and exerted a chemokine-like arrest function as a noncognate CXCR2 ligand. Blocking classical CXCR2 agonists (CXCL1/CXCL8) failed to interfere with these effects of MIF (FIG. 1a).

Chinese hamster ovary (CHO) transfectants that express the β₂ integrin ligand, ICAM-1 (intercellular adhesion molecule 1), were used to dissect the mechanisms by which MIF promotes integrin-dependent arrest. As quantified under flow conditions, the exposure of CHO transfectants to MIF for 2 h resulted in its surface presentation (FIG. 1b) and, like exposure of the transfectants to CXCL8, increased monocytic cell arrest (FIG. 1c). This effect was fully sensitive to PTX and an antibody to β₂ integrin (FIG. 1c), confirming a role of G_{αi} in β₂ integrin-mediated arrest induced by MIF. Primary monocytes and MonoMac6 cells express both CXCR1 and CXCR2 (Weber, K. S., et al. (1999) Eur. J. Immunol. 29, 700-712). Whereas blocking CXCR1 had no effect, blocking CXCR2 substantially but not fully impaired MIF-triggered and CXCL8-triggered monocytic cell arrest. Addition of antibodies to both CXCR1 and CXCR2 completely inhibited the arrest functions of MIF or CXCL8 (FIG. 1d). The use of antibodies to CD74 implicated this protein, along with CXCR2, in MIF-induced arrest (FIG. 1d). Spontaneous arrest was unaffected (data not shown). Thus, CXCR2 assisted by CD74 mediates MIF-induced arrest.

### MIF Induced T-Cell Arrest Through CXCR4

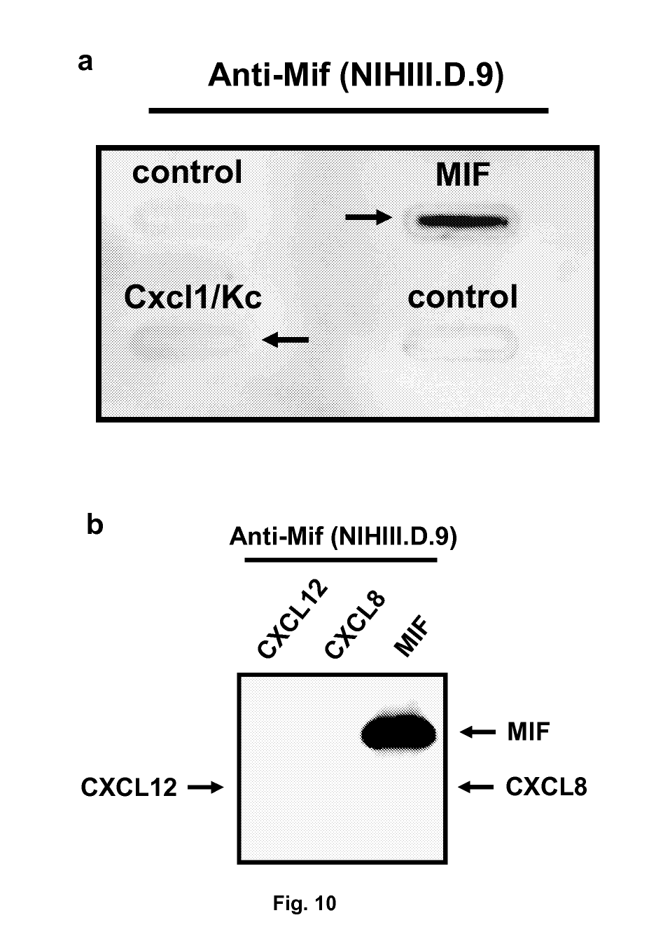

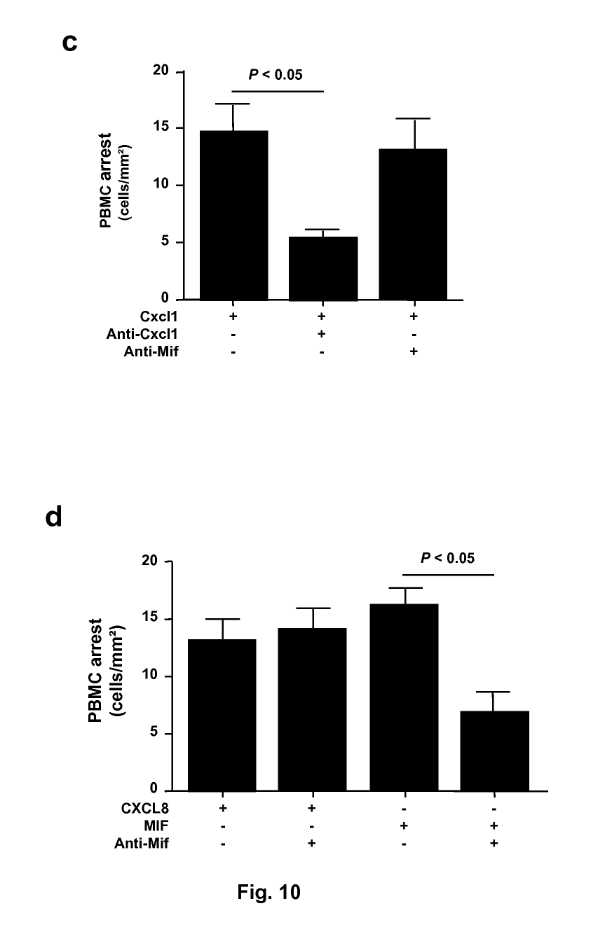

Either MIF or CXCL12 immobilized on aortic endothelial cells triggered the arrest of primary human effector T cells (FIG. 1e). MIF-induced, but not spontaneous, T-cell arrest was sensitive to PTX and was inhibited by an antibody to CXCR4 (FIG. 1e). Although less pronounced than in monocytes expressing CXCR2 (FIG. 1d), presentation of MIF (or CXCL12) on CHO transfectants expressing ICAM-1 elicited α_{L}β₂-dependent arrest of Jurkat T cells, an effect mediated by CXCR4 (FIG. 10.

Ectopic expression of CXCR2 in Jurkat T cells increased MIF-triggered arrest (FIG. 1g), corroborating the idea that CXCR2 imparts responsiveness to MIF in leukocytes. L1.2 pre-B lymphoma transfectants expressing CXCR1, CXCR2 or CXCR3, and controls using cells expressing endogenous CXCR4 only were used in the presence of the CXCR4 antagonist AMD3465. MIF triggered the arrest of CXCR2 transfectants and CXCR4-bearing controls on endothelial cells with a similar efficacy to that of the canonical ligands CXCL8 and CXCL12, whereas CXCR1 and CXCR3 transfectants were responsive to CXCL8 and CXCL10, respectively, but not to MIF (FIG. 1h). This data established that CXCR2 and CXCR4, but not CXCR1 or CXCR3, support MIF-induced arrest.

### Example 3

### MIF-Induced Leukocyte Chemotaxis Through CXCR2/4 Activation

Chemokines have been eponymously defined as inducers of chemotaxis (Baggiolini, M., et al. (1994) Adv. Immunol. 55, 97-179; Weber, C., et al. (2004) Arterioscler. Thromb. Vasc. Biol. 24, 1997-2008). Paradoxically, MIF was initially thought to interfere with ‘random’ migration (Calandra, T., et al. (2003) Nat. Rev. Immunol. 3, 791-800). Although this may be attributable to active repulsion or desensitization of directed emigration, specific mechanisms evoked by MIF to regulate migration remain to be clarified. As cell activation by MIF may rather stimulate migration (Schrans-Stassen, B. H. G. J., et al. (2005) Antioxid. Redox Signal. 7, 1211-1216), our results showing that MIF induced G_{αi}-mediated functions of CXCR2 and CXCR4 prompted us to test if MIF directly elicits leukocyte chemotaxis through these receptors.

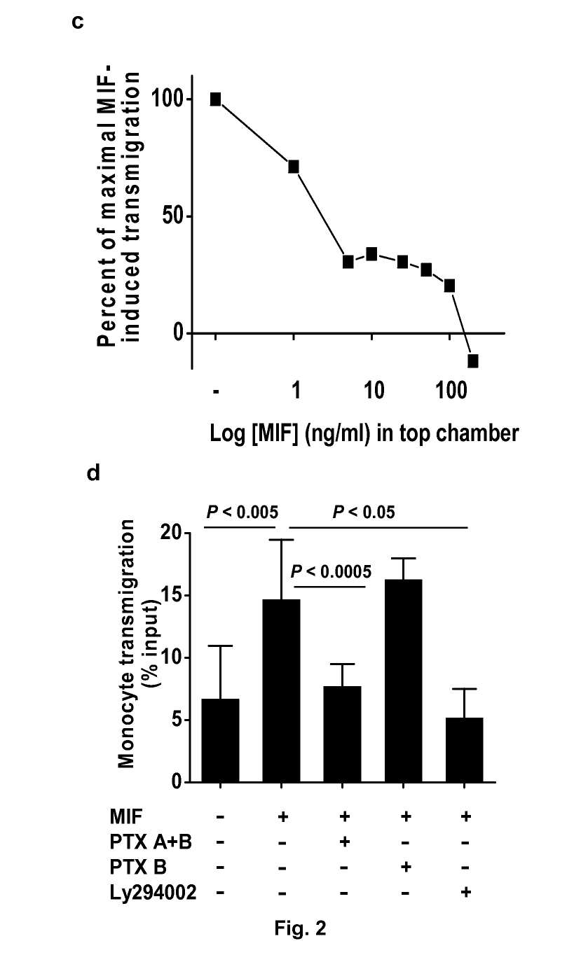

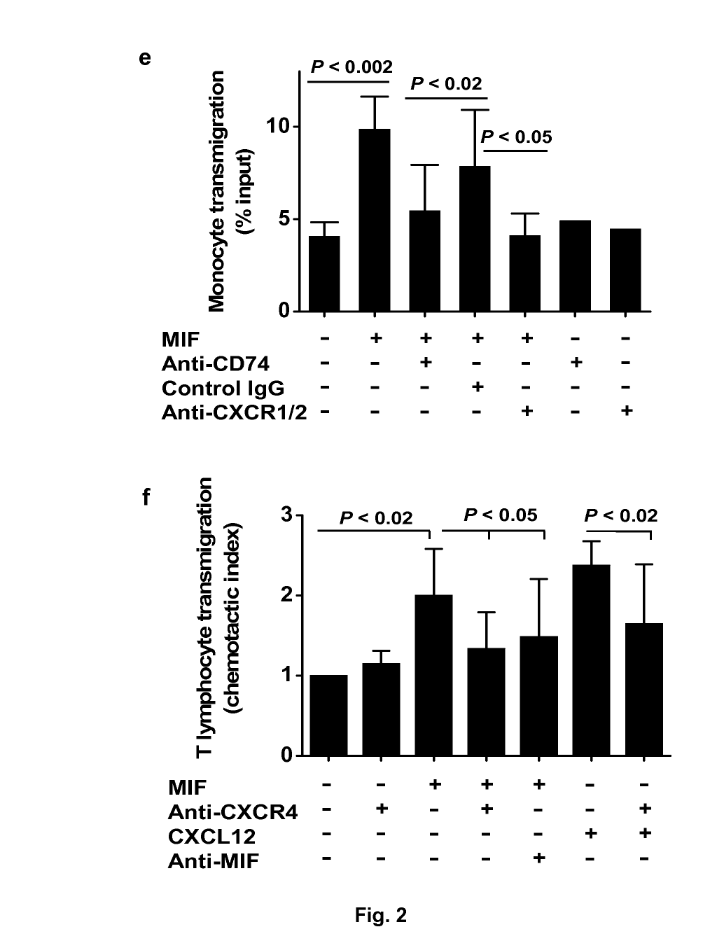

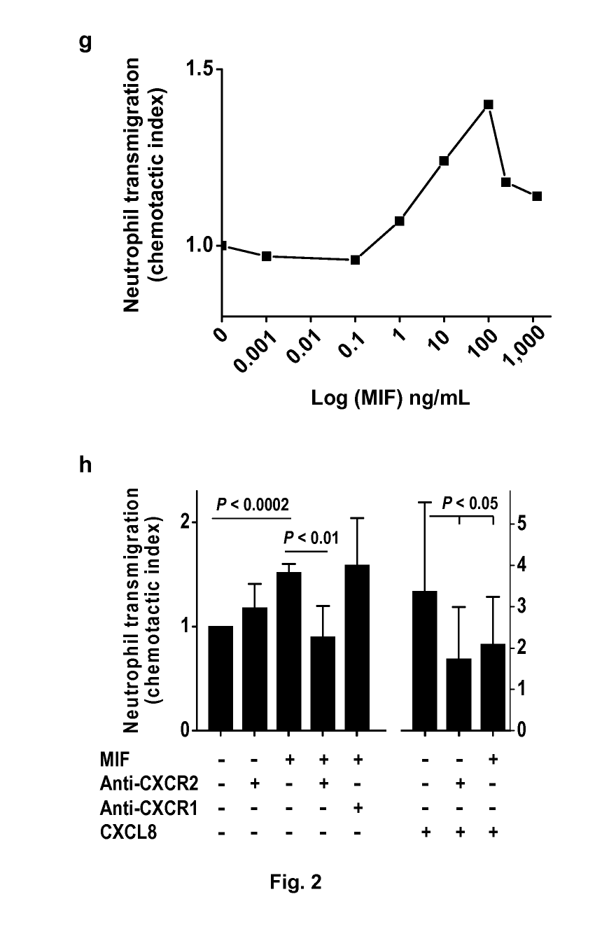

Using a transwell system, the promigratory effects of MIF and CXCL8 were compared on primary human peripheral blood mononuclear cell-derived monocytes. CCL2 was also used as a prototypic chemokine for monocytes. Similar to CXCL8 and CCL2, adding MIF to the lower chamber induced migration, which followed a bell-shaped dose-response curve typical for chemokines, with an optimum at 25-50 ng/ml, albeit with a lower peak migratory index (FIG. 2a). Heat treatment or a neutralizing antibody to MIF abolished MIF-induced transmigration. In contrast, isotype-matched immunoglobulin (IgG) had no effect (FIG. 2b). When added to the upper chamber, MIF dose-dependently desensitized migration toward MIF in the lower chamber (FIG. 2c) but did not elicit migration when present in the upper chamber only (data not shown), suggesting that MIF evokes true chemotaxis rather than chemokinesis. Consistent with G_{αi}-dependent signaling through phosphoinositide-3-kinase, MIF-induced monocyte chemotaxis was sensitive to PTX and abrogated by Ly294002 (FIG. 2d). Both CXCR2 and CD74 specifically contributed to MIF-triggered monocyte chemotaxis (FIG. 2e). The role for CXCR2 was confirmed by showing MIF-mediated cross-desensitization of CXCL8-induced chemotaxis in CXCR2-transfected L1.2 cells (data not shown). The chemotactic activity of MIF was verified in RAW264.7 macrophages and THP-1 monocytes (data not shown). These data demonstrate that MIF triggers monocyte chemotaxis through CXCR2.

To substantiate functional MIF-CXCR4 interactions, the transmigration of primary CD3⁺ T lymphocytes devoid of CXCR1 and CXCR2 was evaluated. Similar to CXCL12, a known CXCR4 ligand and T-cell chemoattractant, MIF dose-dependently induced transmigration, a process that was chemotactic and transduced through CXCR4, as shown by antibody blockade and cross-desensitization of CXCL12 (FIG. 20. Thus, MIF elicits directed T-cell migration through CXCR4. In primary human neutrophils, a major cell type bearing CXCR2, MIF exerted CXCR2- but not CXCR1-mediated chemotactic activity, exhibiting a bell-shaped dose-response curve and cross-densensitizing CXCL8 (FIG. 2g,h). The moderate chemotactic activity of neutrophils towards MIF is likely to be related to an absence of CD74 on neutrophils, as its ectopic expression in CD74⁻ promyelocytic HL-60 cells enhanced MIF-induced migration (Supplementary FIG. 2). Although MIF, like other CXCR2 ligands, functions as an arrest chemokine (Huo, Y., et al. (2001) J. Clin. Invest. 108, 1307-1314; Weber, K. S., et al. (1999) Eur. J. Immunol. 29, 700-712), this data revealed that MIF also has appreciable chemotactic properties on mononuclear cells and neutrophils.

### Example 4

### MIF Triggers Rapid Integrin Activation and Calcium Flux

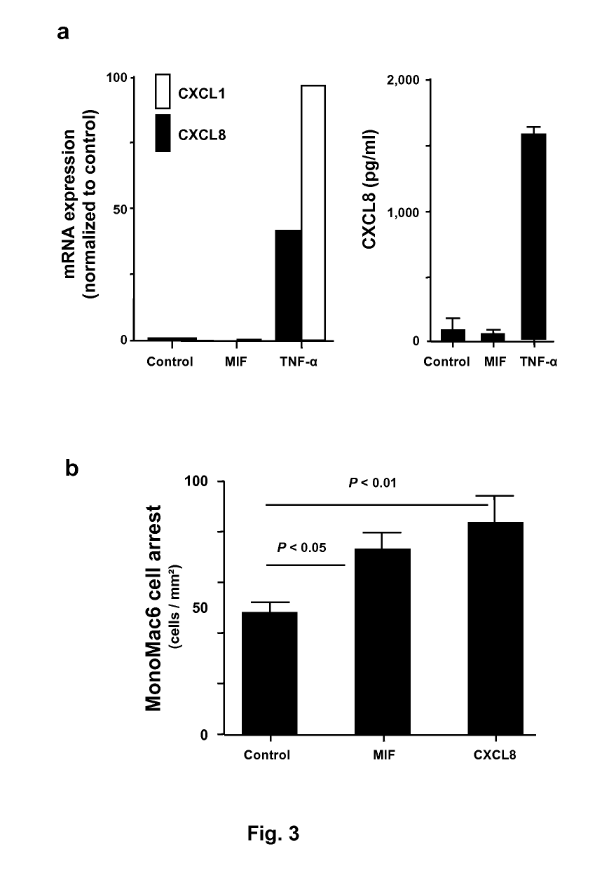

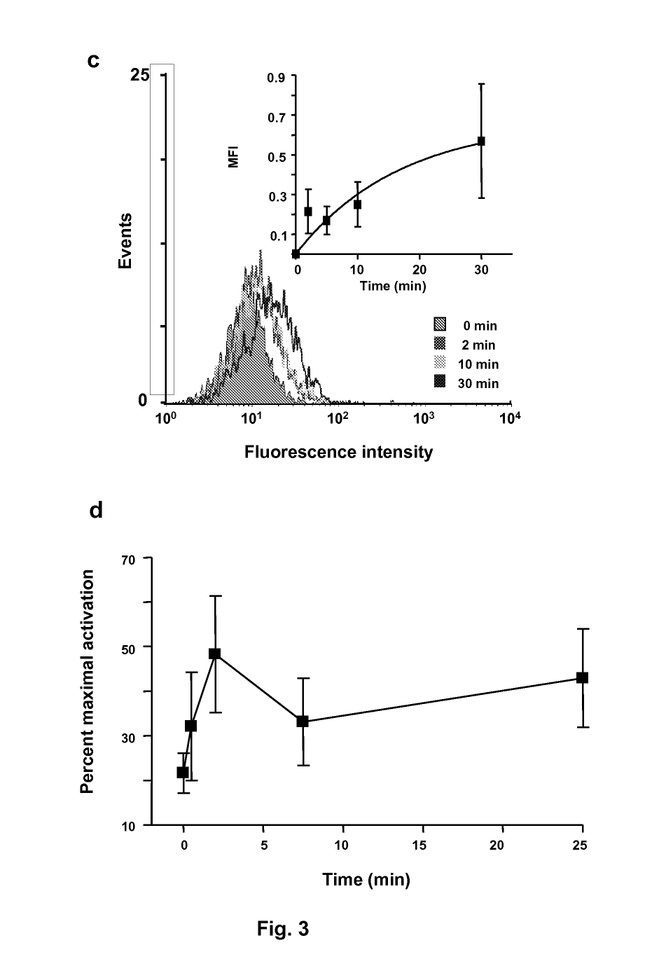

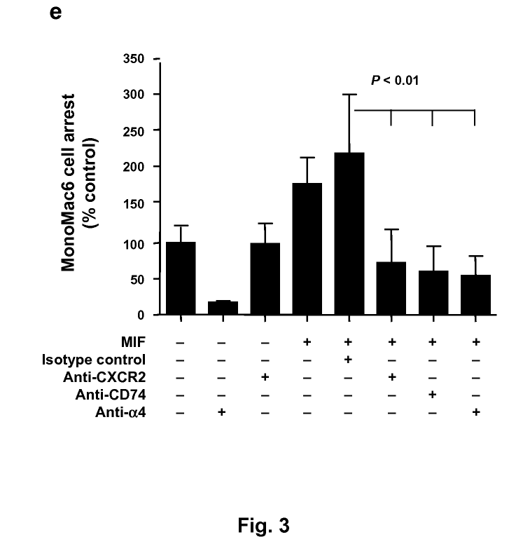

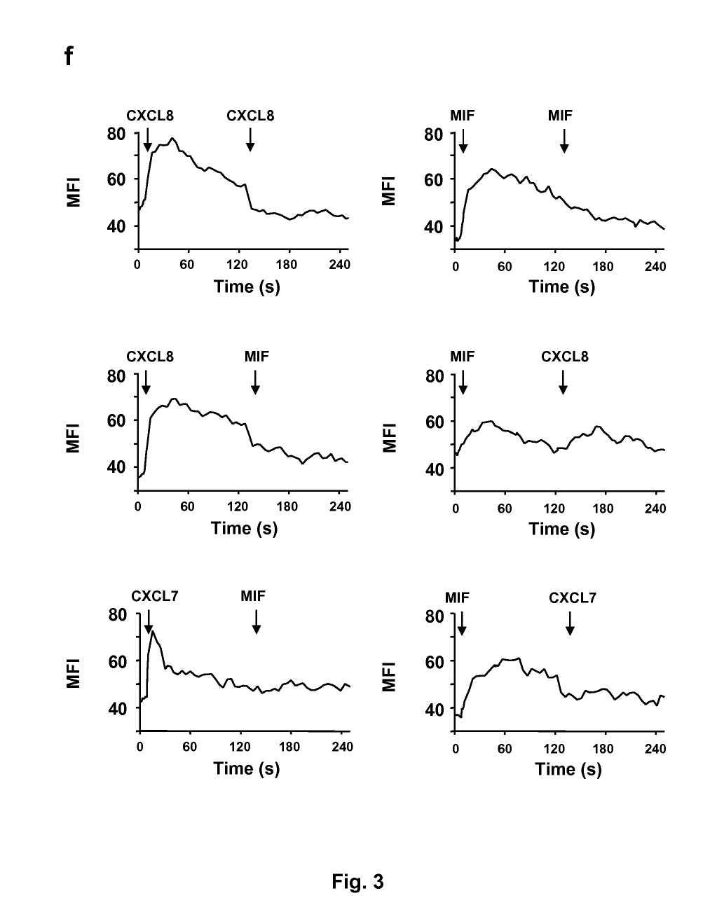

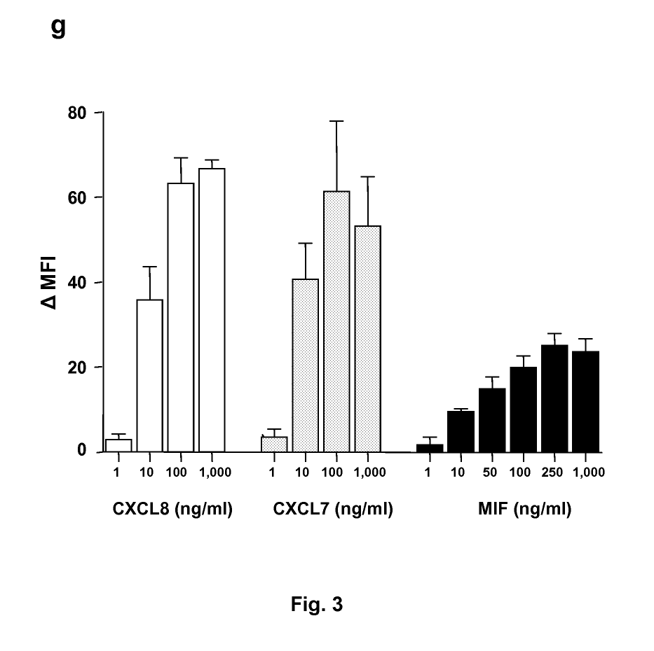

Arrest functions of MIF may reflect direct MIF/CXCR signaling, but it cannot be entirely excluded that MIF induces other arrest chemokines during the time required for MIF immobilization. To consolidate evidence that MIF directly induces leukocyte arrest (FIG. 1), real-time PCR and ELISAs were performed and found that 2-h-long preincubation of human aortic (or venous) endothelial cells with MIF failed to upregulate typical arrest chemokines known to engage CXCR2 (FIG. 3a and data not shown).

Short-term exposure to chemokines present in solution or immobilized in juxtaposition to integrin ligands (for example, vascular cell adhesion molecule (VCAM)-1) can rapidly upregulate integrin activity, which mediates leukocyte arrest (Laudanna, C., et al. (2006) Thromb. Haemost. 95, 5-11). This is accomplished by clustering (for example, α₄α₁) or conformational changes (for example, α_{L}β₂) immediately preceding ligand binding. Stimulation of monocytic cells with ME (or CXCL8) for 1-5 min triggered α_{L}β₂-dependent arrest on CHO/ICAM-1 cells (FIG. 3b). To obtain evidence for a direct stimulation of monocyte integrins, the reporter antibody 327C, which recognizes an extended high-affinity conformation of α_{L}β₂, was used (Shamri, R., et al. (2005) Nat. Immunol. 6, 497-506). These assays revealed that α_{L}β₂ activation in MonoMac6 cells (FIG. 3c) and human blood monocytes (FIG. 3d) occurred as early as 1 min after exposure to MIF and persisted over 30 min. To evaluate whether MIF's effects were restricted to α_{L}β₂, α₄β₁-dependent monocytic cell arrest on VCAM-1 was studied. Exposure to MIF for 1-5 min induced marked arrest, which was mediated by CXCR2, CD74 and α₄β₁ (FIG. 3e). Similarly to the effect of CXCL12, stimulation of Jurkat T cells with MIF for 1-5 min triggered CXCR4-dependent adhesion on VCAM-1 (data not shown).

As CXCR2 can mediate increases in cytosolic calcium elicited by CXCL8 (Jones, S. A., et al. (1997) J. Biol. Chem. 272, 16166-16169), the ability of MIF to stimulate calcium influx and desensitize CXCL8 signals was tested. Indeed, like CXCL8, MIF induced calcium influx in primary human neutrophils and desensitized calcium transients in response to either CXCL8 or MIF (FIG. 31), confirming that MIF activates GPCR/G_{αi} signaling. The partial desensitization of CXCL8 signaling by MIF seen in neutrophils parallels findings with other CXCR2 ligands (Jones, S. A., et al. (1997) J. Biol. Chem. 272, 16166-16169) and reflects the presence of CXCR1. In L1.2 transfectants expressing CXCR2, MIF fully desensitized CXCL8-induced calcium influx (data not shown), and in neutrophils, MIF desensitized transients induced by the selective CXCR2 ligand CXCL7 (and CXCL7 desensitized transients induced by MIF) (FIG. 31). In CXCR2 transfectants, MIF dose-dependently induced calcium influx, and was slightly less potent and effective than CXCL8 or CXCL7 (FIG. 3g). In conclusion, MIF acted on CXCR2 and CXCR4 to elicit rapid integrin activation and calcium influx.

### Example 5

### MIF Interacts with CXCR2 and CXCR4

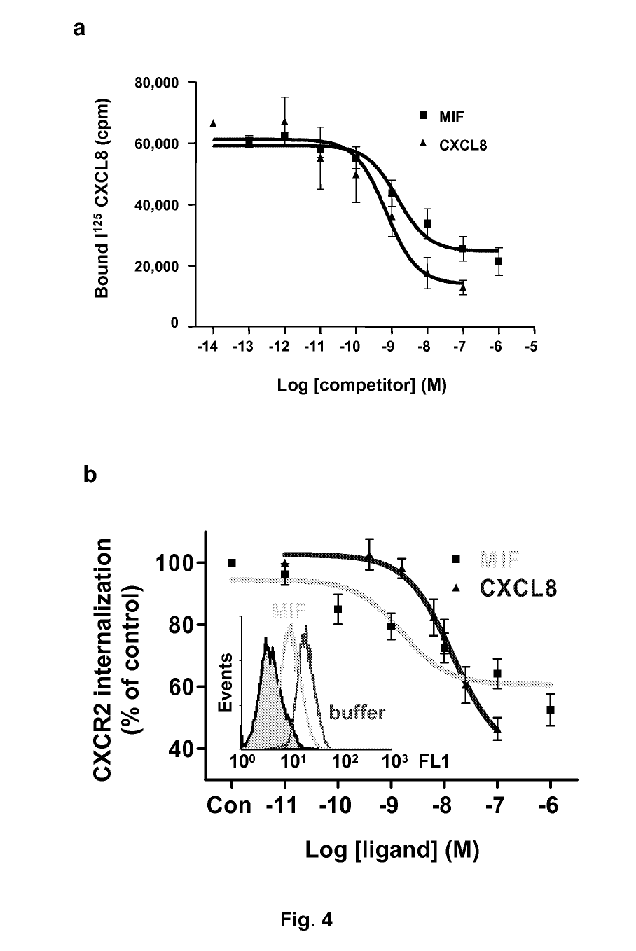

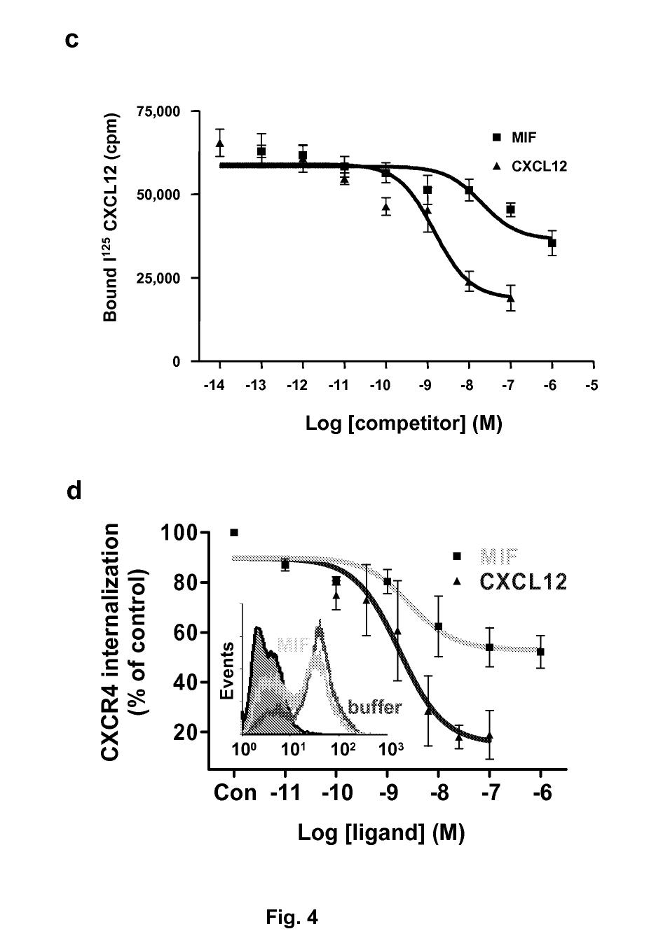

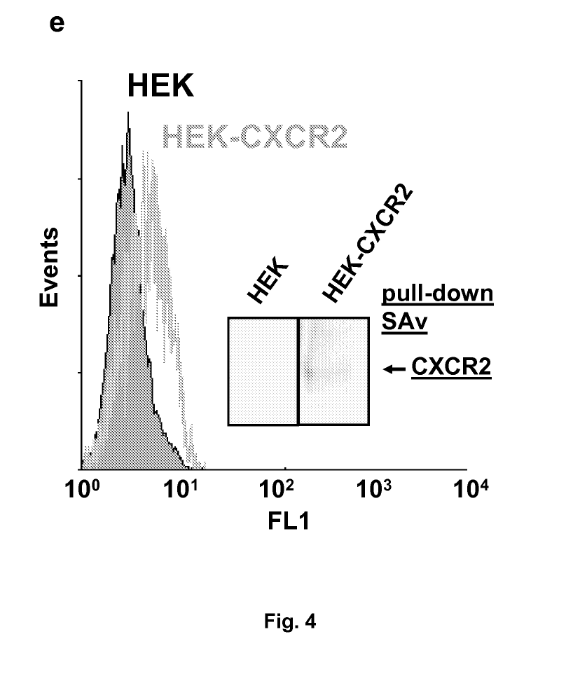

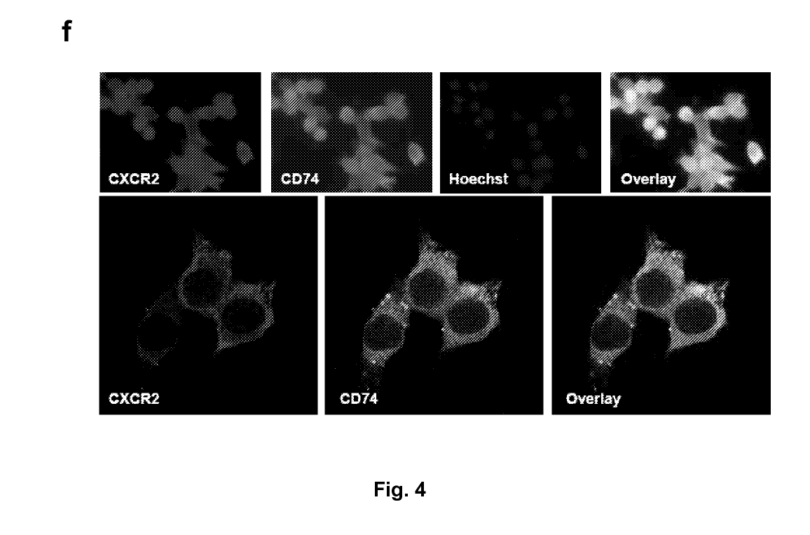

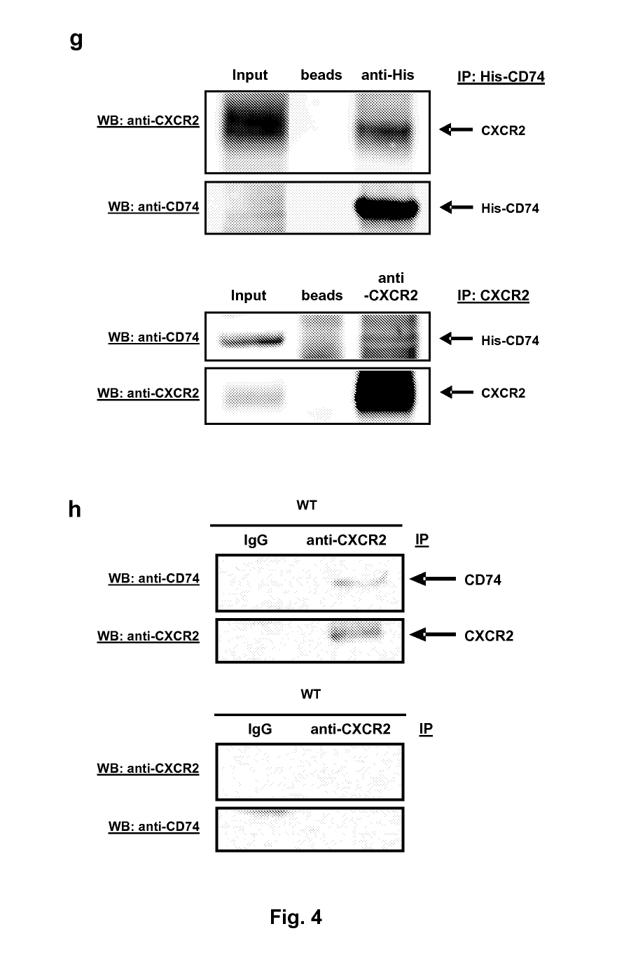

To assess the physical interactions of MIF with CXCR2 and CXCR4, we performed receptor-binding competition and internalization studies. In HEK293 cells ectopically expressing CXCR2, MIF strongly competed with ¹²⁵I-labeled CXCL8 for CXCR2 binding under equilibrium conditions. Binding of the CXCL8 tracer to CXCR2 was inhibited by MIF with an effector concentration for half-maximum response (EC₅₀) of 1.5 nM (FIG. 4a). The affinity of CXCR2 for MIF (K_{d}=1.4 nM) was close to that for CXCL8 (K_{d}=0.7 nM) and within the range of the MIF concentration that induced optimal chemotaxis (2-4 nM). To confirm binding to CXCR2, we used a receptor internalization assay that reports specific receptor-ligand interactions. FACS analysis of surface CXCR2 on stable HEK293 transfectants showed that MIF induced CXCR2 internalization with a dose response resembling that of CXCL8 (FIG. 4b). Comparable data was obtained in CXCR2-transfected RAW264.7 macrophages (inset in FIG. 4b, and data not shown).

To verify an interaction of MIF with CXCR4, receptor-binding studies were performed in Jurkat T cells, which endogenously express CXCR4. MIF competed with ¹²⁵I-labeled CXCL12 for CXCR4 binding (K_{d} for CXCL12=1.5 nM; EC₅₀=19.9 nM, K_{d} for MIF=19.8 nM) (FIG. 4c). The K_{d} was in accordance with MIF concentrations that induce T-cell chemotaxis. Consistently, MIF, like CXCL12, elicited CXCR4 internalization in a dose-dependent fashion (FIG. 4d). MIF-induced internalization of CXCR2 and CXCR4 was specific to these receptors, as MIF, unlike the cognate ligand CCL5, was unable to induce CCR5 internalization in L1.2 CCR5 transfectants (data not shown).

To corroborate its interactions with CXCRs, MIF was labeled with biotin or fluorescein, which, in contrast to iodinated MIF, allows for direct receptor-binding assays. CXCR2 transfectants, but not vector controls, supported direct binding of labeled MIF, as evidenced by flow cytometry (FIG. 4e), pull down with streptavidin beads (inset in FIG. 4e) and fluorescence microscopy (data not shown). In addition, the specific binding of fluorescein-MIF to CXCR4-bearing Jurkat cells was inhibited by the CXCR4 antagonist AMD3465 (data not shown).

### Complex Formation Between CXCR2 and CD74

CD74 has been implicated as a MIF-binding protein. Therefore our data suggests the possibility that a functional MIF receptor complex involves both GPCRs and CD74. To investigate this theory, the colocalization of endogenous CD74 and CXCR2 was visualized using confocal fluorescence microscopy in RAW264.7 macrophages expressing human CXCR2. Using this technique, prominent colocalization was observed in a polarized pattern in ˜50% of cells (FIG. 41).

In addition, coimmunoprecipitation assays revealed that CXCR2 physically interacts with CD74. CXCR2/CD74 complexes were detected in HEK293 cells stably overexpressing CXCR2 and transiently expressing His-tagged CD74. These complexes were observed by precipitation with an antibody to CXCR2 and by detecting coprecipitated CD74 by western blot against the His-tag. Coprecipitation was also seen when the order of the antibodies used was reversed (FIG. 4g). Complexes were also detected with CD74 in L1.2 transfectants stably expressing human CXCR2, as assessed by coimmunoprecipitation with an antibody to CXCR2. In contrast, no complexes were observed with L1.2 controls or the isotype control (FIG. 4h). This data suggested a model in which CD74 forms a signaling complex with CXCR2 to mediate MIF functions.

### Example 6

### CXCR2 Mediates MIF-Induced Monocyte Arrest in Arteries

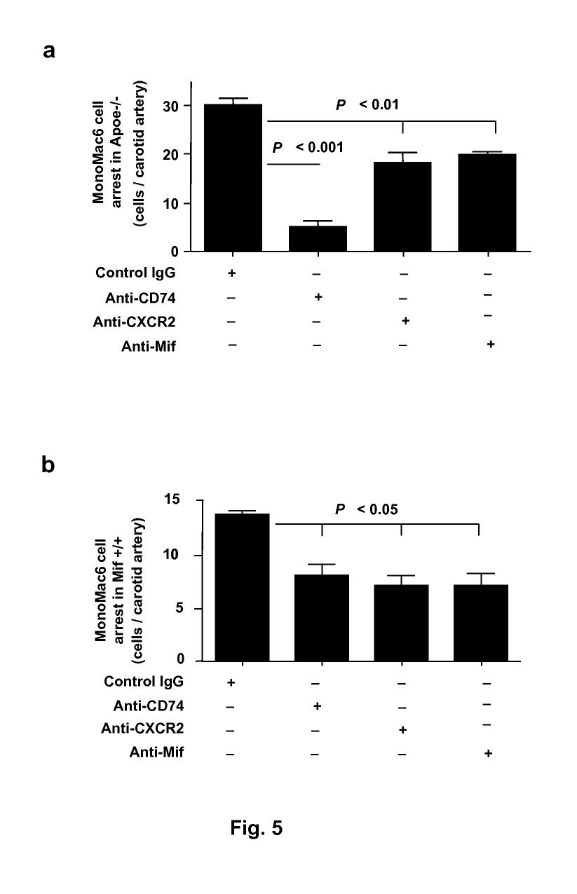

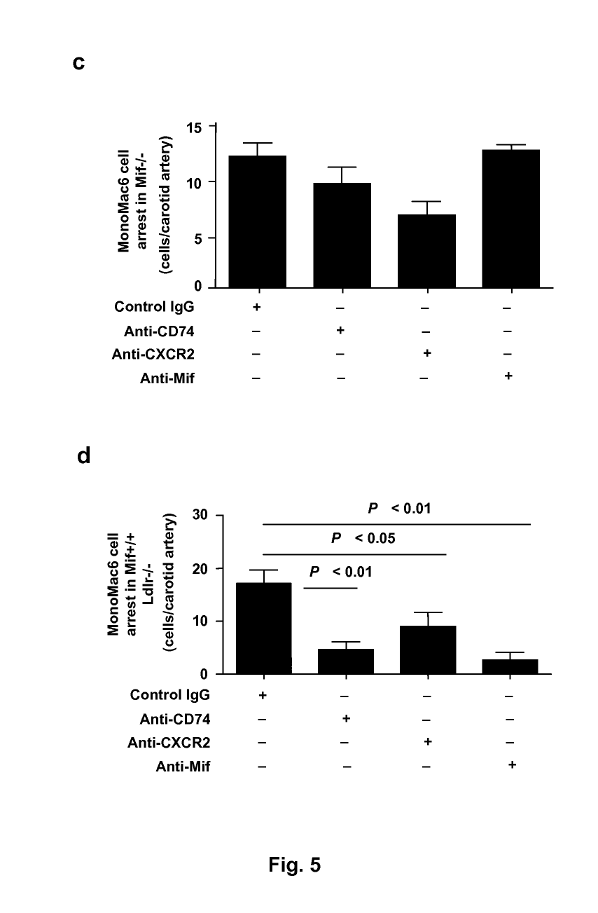

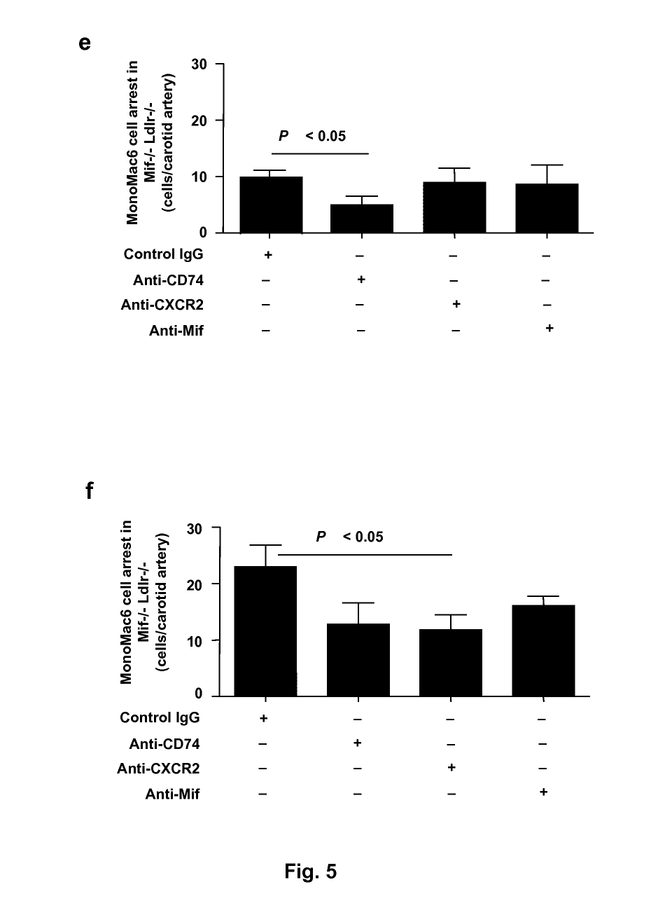

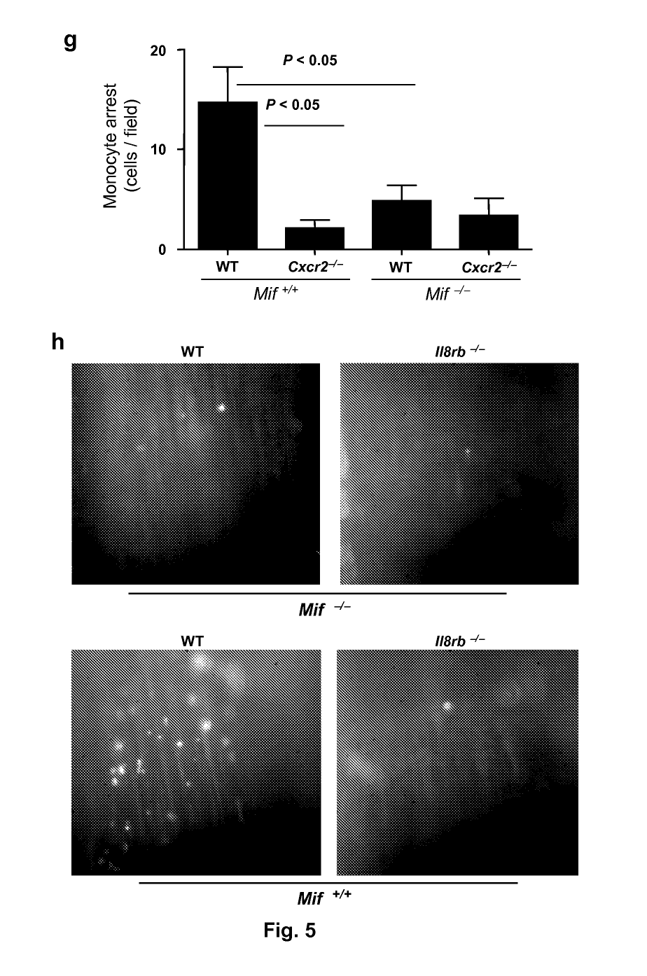

MIF promotes the formation of complex plaques with abundant cell proliferation, macrophage infiltration and lipid deposition (Weber, C., et al. (2004) Arterioscler. Thromb. Vasc. Biol. 24, 1997-2008; Morand, E. F., et al. (2006) Nat. Rev. Drug Discov. 5, 399-410). This has been related to the induction of endothelial MIF by oxLDL, triggering monocyte arrest (Schober, A., et al. (2004) Circulation 109, 380-385). The CXCR2 ligand CXCL1 can also elicit α₄β₁-dependent monocyte accumulation in ex vivo-perfused carotid arteries of mice with early atherosclerotic endothelium (Huo, Y., et al. (2001) J. Clin. Invest. 108, 1307-1314). This system was used to test whether MIF acts via CXCR2 to induce recruitment. Monocyte arrest in carotid arteries of Apoe^{-/-} mice fed a high-fat diet was inhibited by antibodies to CXCR2, CD74 or MIF (FIG. 5a and data not shown), indicating that MIF contributed to atherogenic recruitment via CXCR2 and CD74. Following the blockade of MIF, CXCR2 and CD74 for 24 h, a similar pattern was observed for monocyte arrest in arteries of wild-type mice treated with tumor necrosis factor (TNF)-α, mimicking acute vascular inflammation (FIG. 5b). In arteries of TNF-α-treated Mif^{-/-} mice, inhibitory effects on CD74 were attenuated and blocking MIF was ineffective, whereas there was residual CXCR2 inhibition, implying the involvement of other inducible ligands (FIG. 5c). Compared to the effect of MIF deficiency observed with TNF-α stimulation, monocyte accumulation was more clearly impaired by MIF deficiency in arteries of Mif^{-/-} Ldlr^{-/-} mice (compared to atherogenic Mif^{+/+} Ldlr^{-/-} mice; FIG. 5d,e). In the absence of MIF, there was no apparent contribution of CXCR2. Moreover, blocking MIF had no effect (FIG. 5d,e). The inhibitory effects of blocking CXCR2 were restored by loading exogenous MIF (FIG. 5f).

To provide further evidence for the idea that CXCR2 is required for MIF-mediated monocyte recruitment in vivo, intravital microscopy was performed on carotid arteries of chimeric wild-type Mif^{+/+} and Mif^{-/-} mice reconstituted with wild-type or Il8rb^{-/-} bone marrow (Il8rb encodes CXCR2; FIG. 5g,h and data not shown). After treatment with TNF-α for 4 h, the accumulation of rhodamine G-labeled leukocytes was attenuated in Mif^{-/-} mice reconstituted with wild-type bone marrow compared to that in wild-type mice reconstituted with wild-type bone marrow. The reduction in leukocyte accumulation due to deficiency in bone marrow CXCR2 was more marked in chimeric wild-type mice than in chimeric Mif^{-/-} mice (FIG. 5g,h and data not shown).

### Example 7

### MIF-Induced Inflammation In Vivo Relies on CXCR2

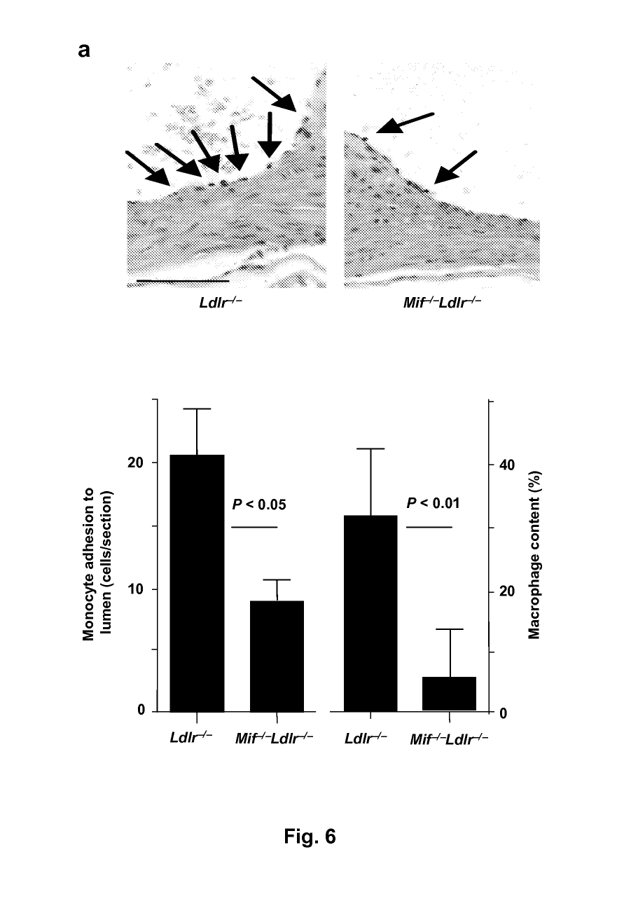

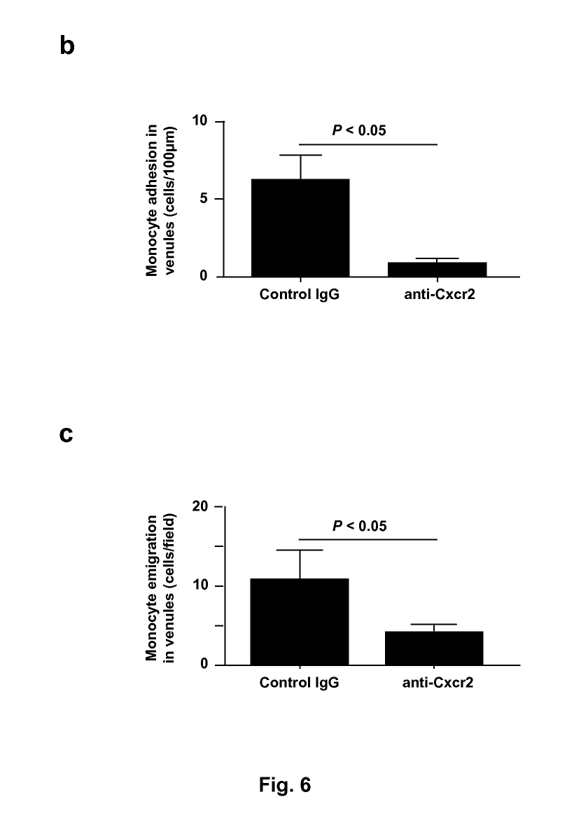

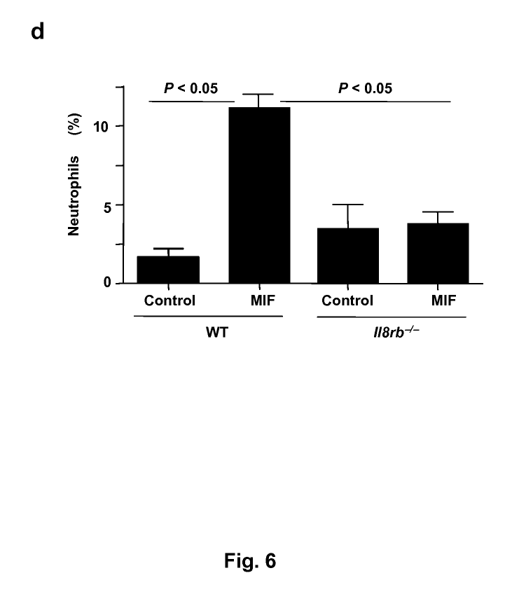

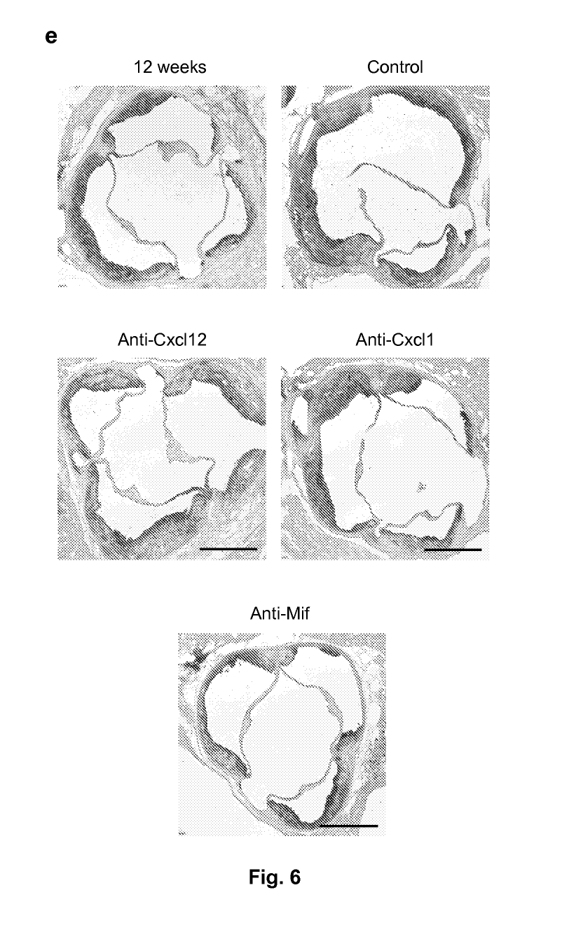

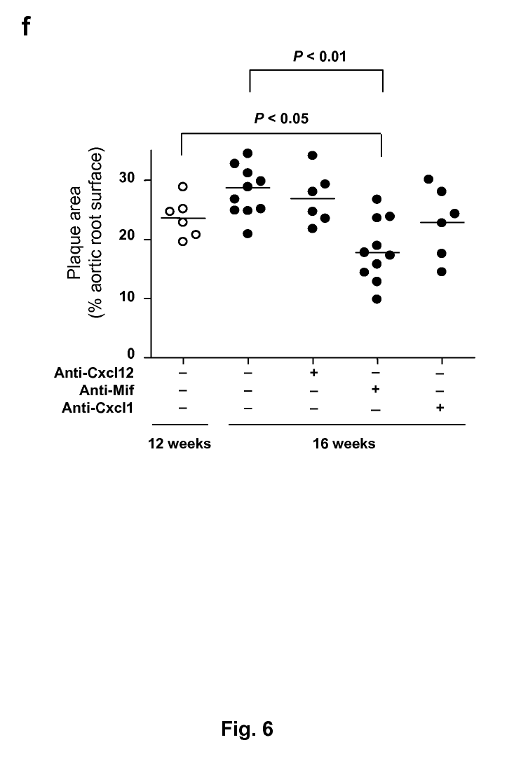

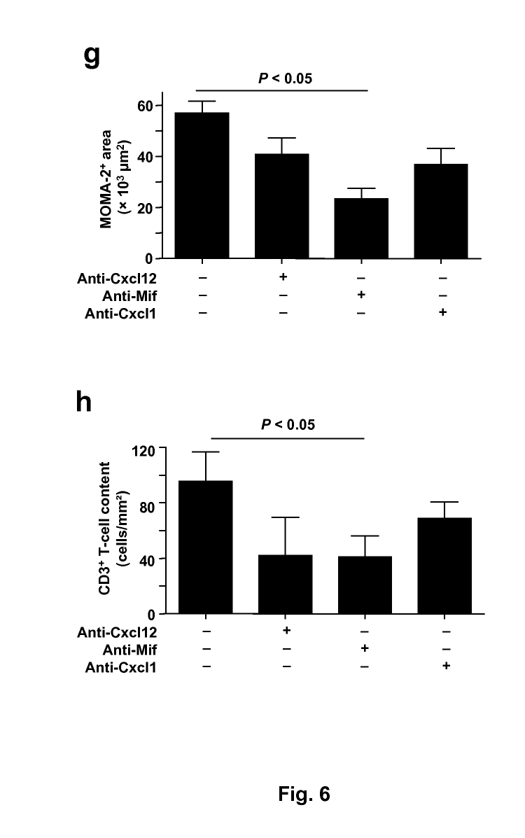

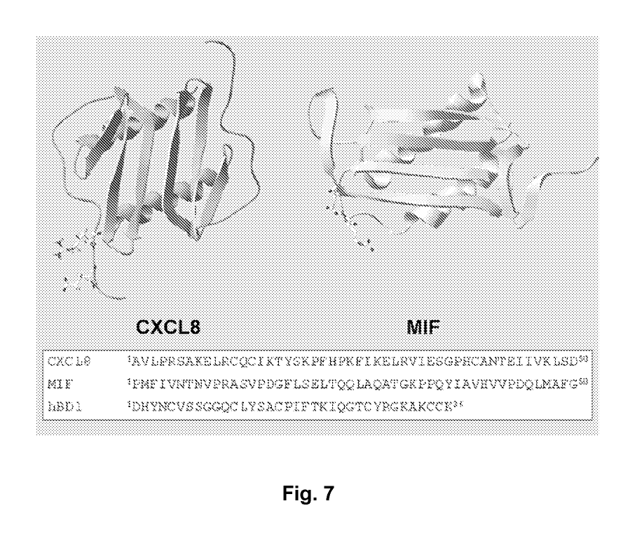

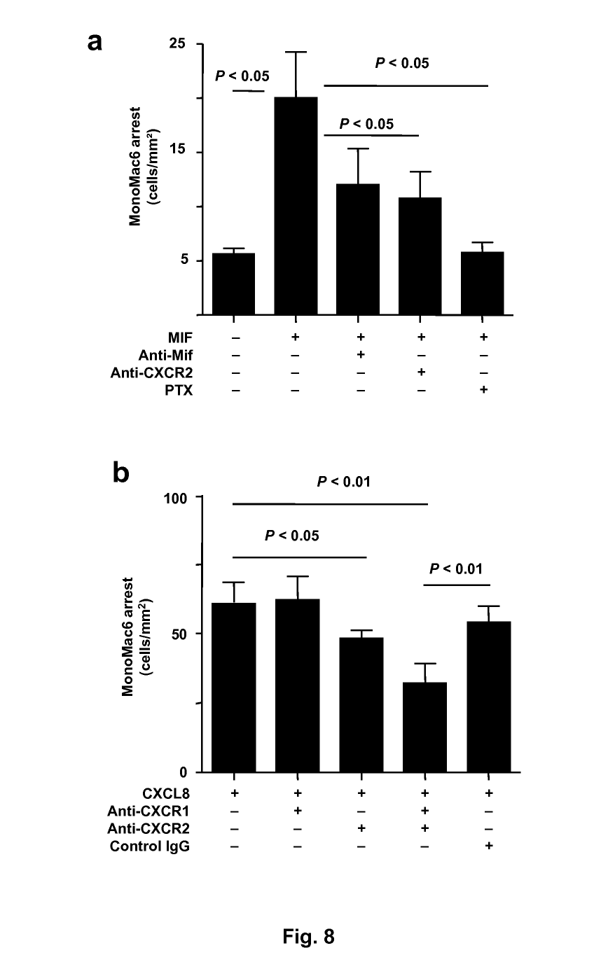

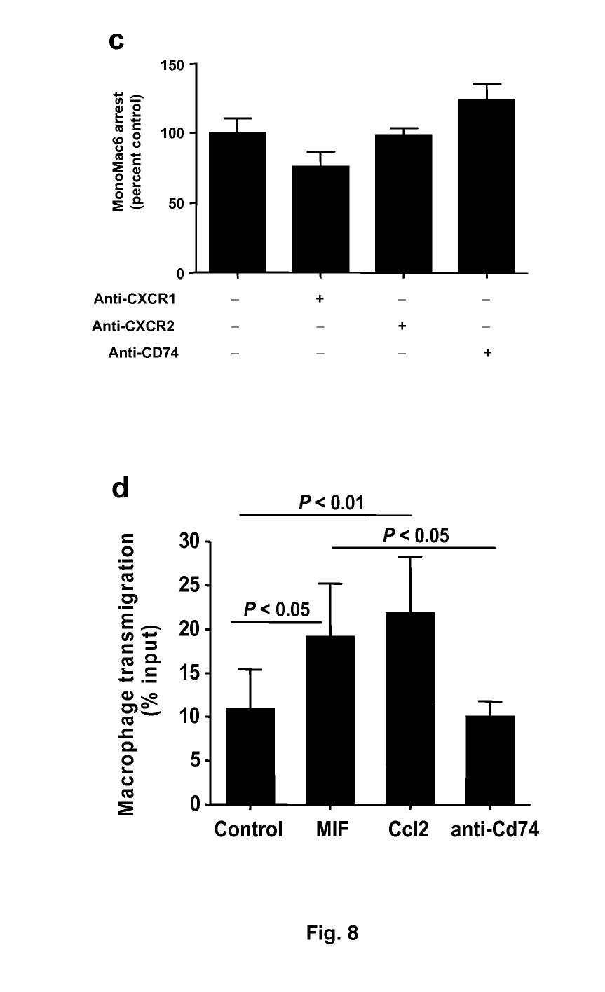

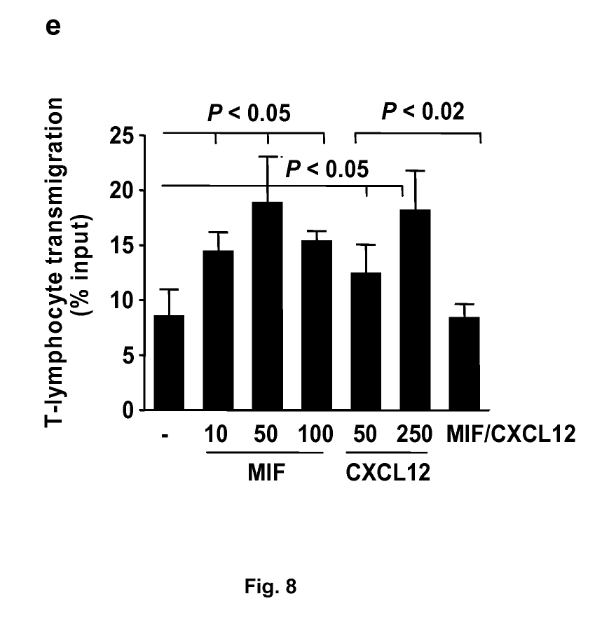

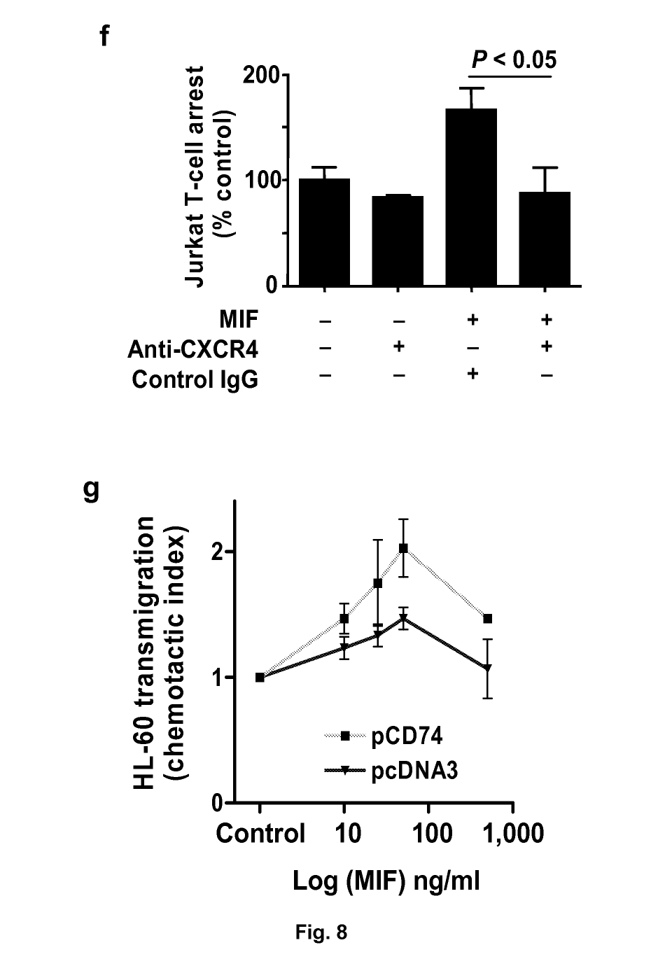

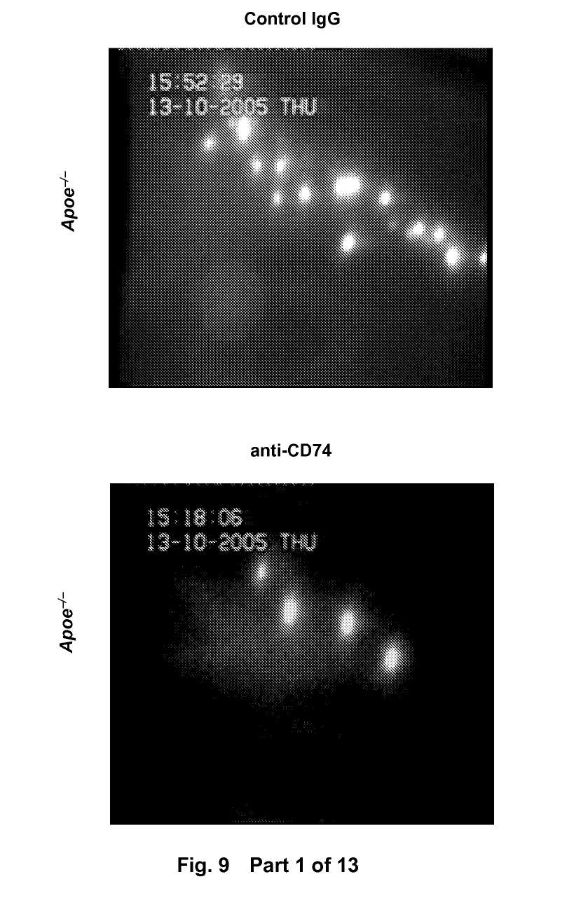

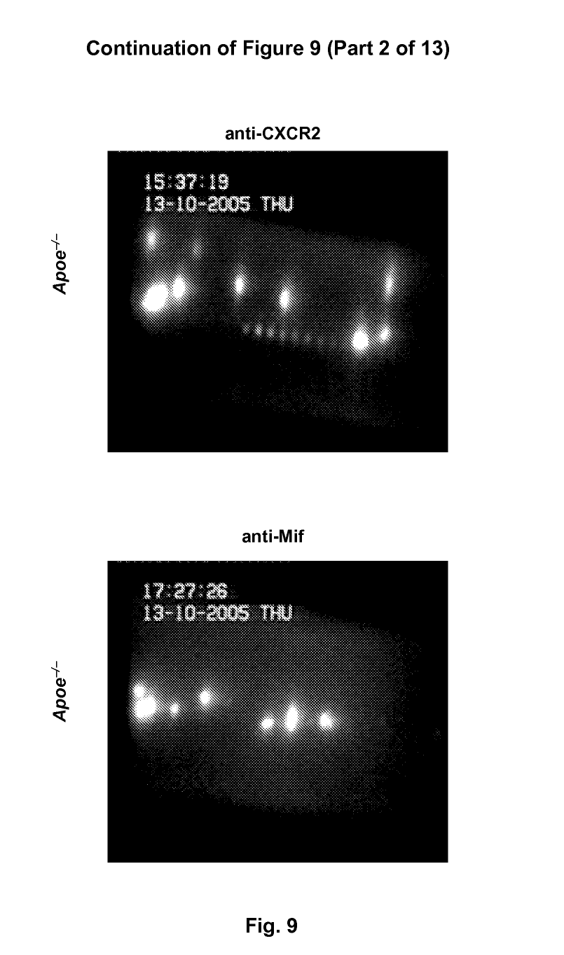

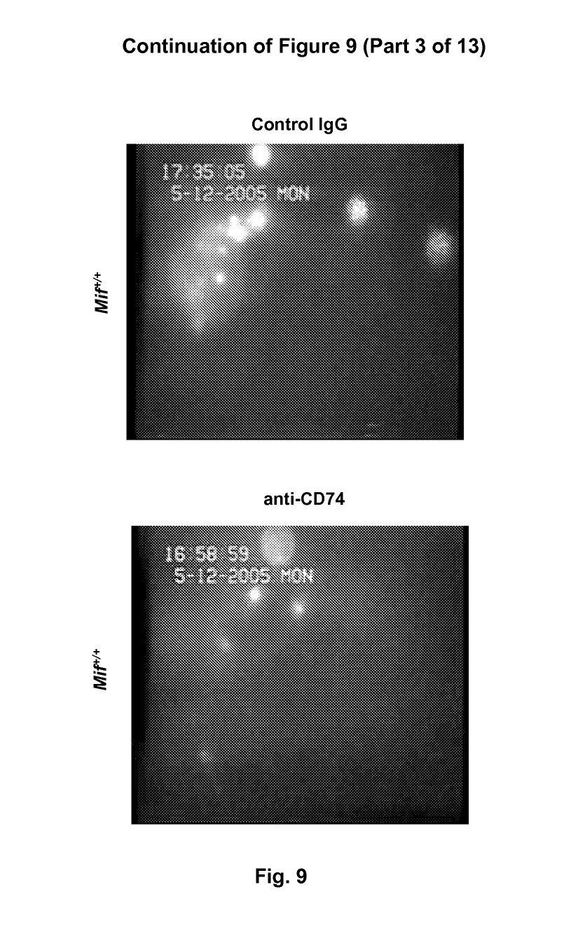

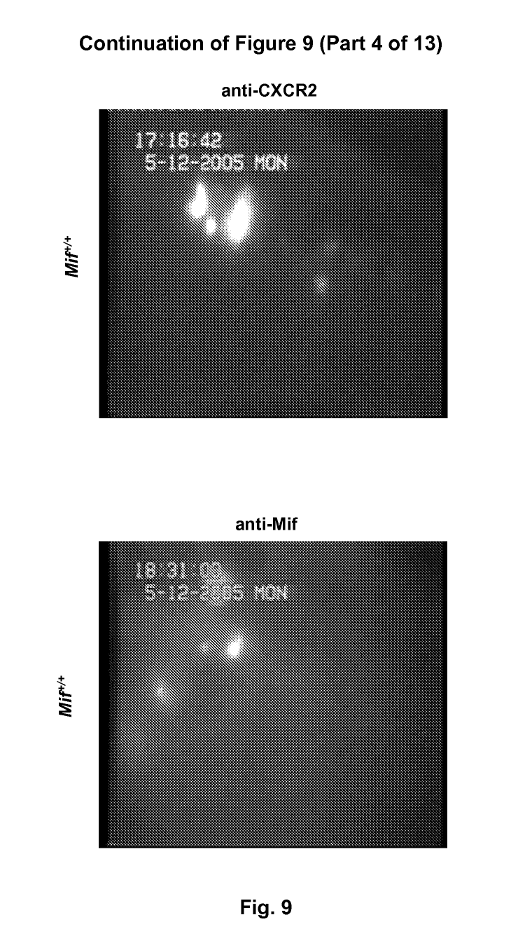

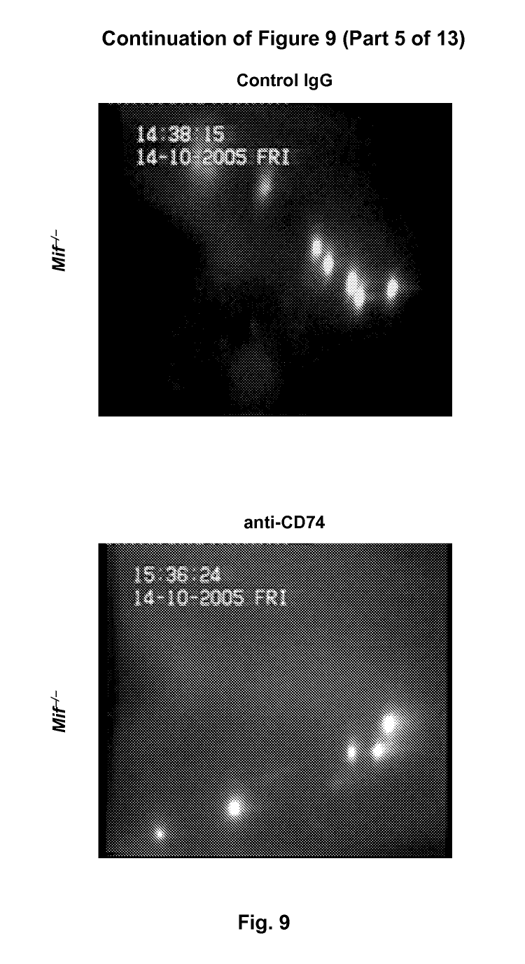

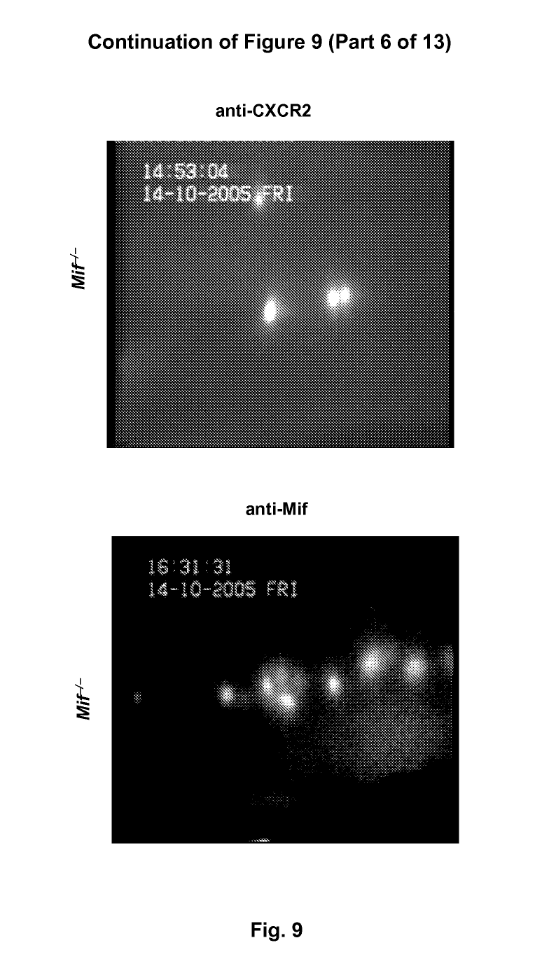

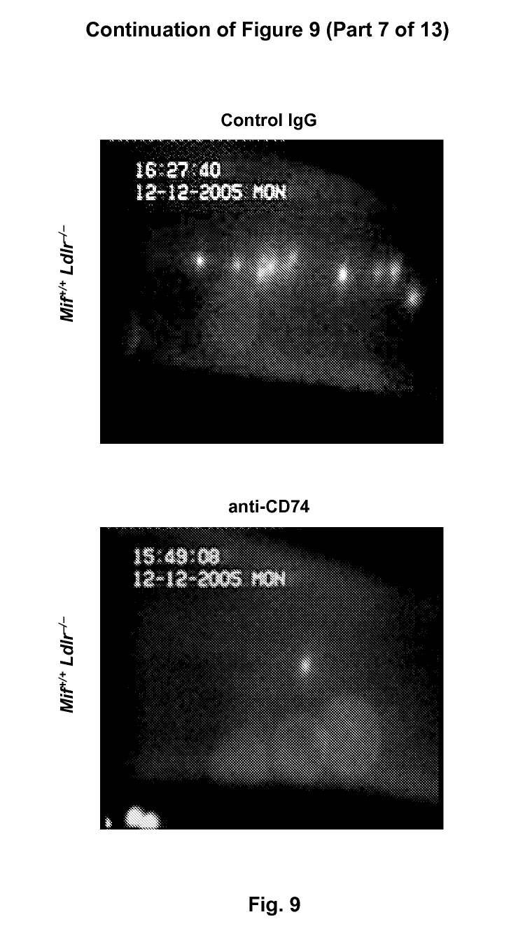

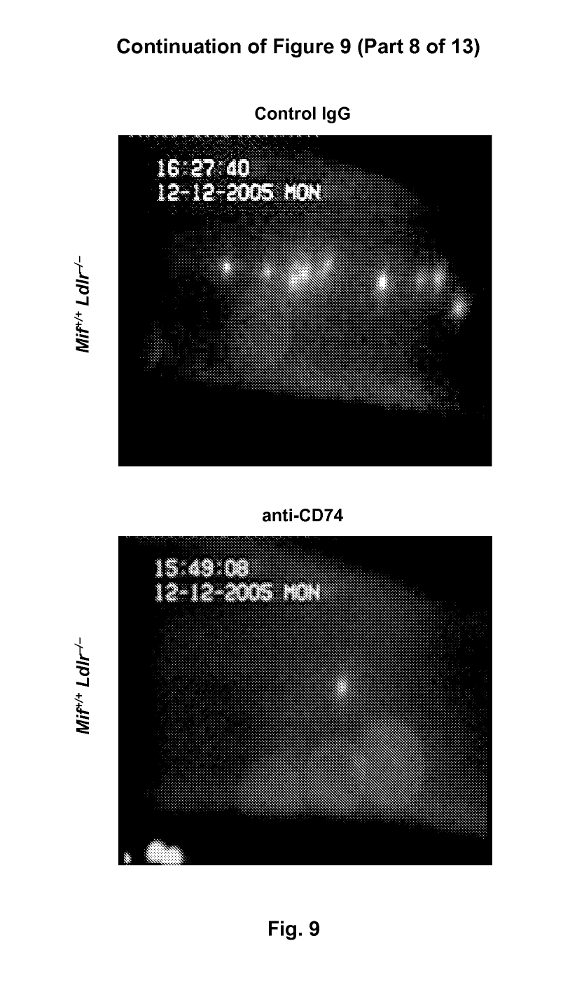

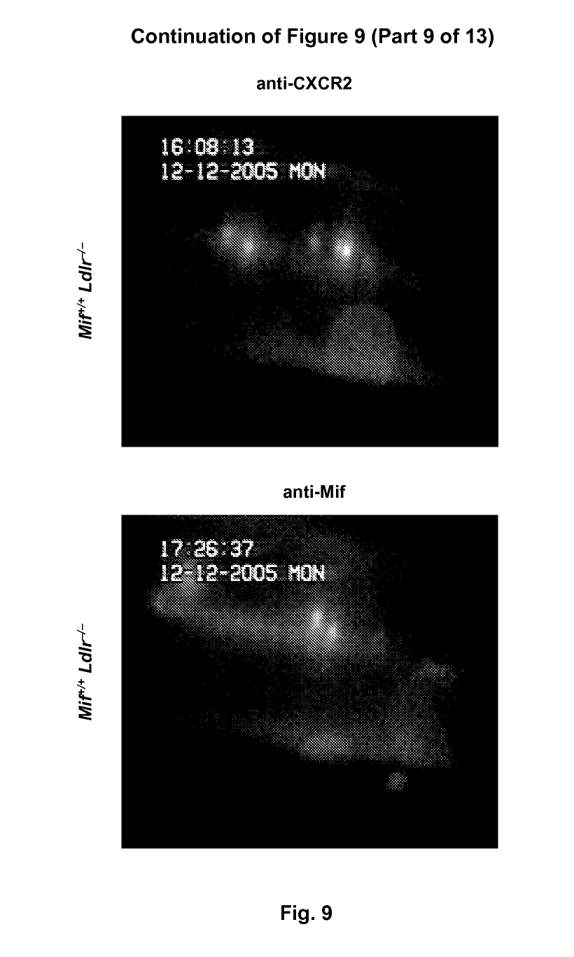

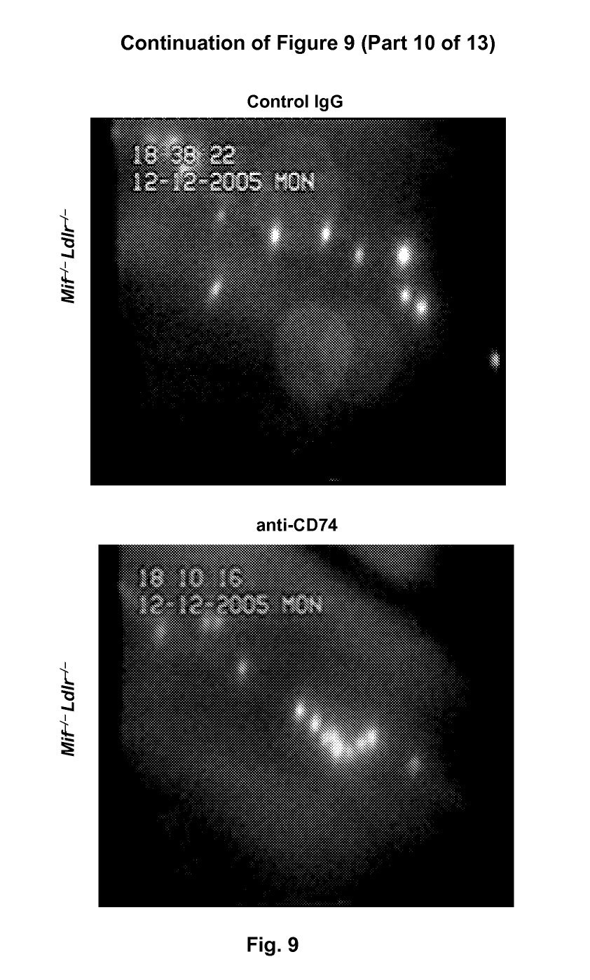

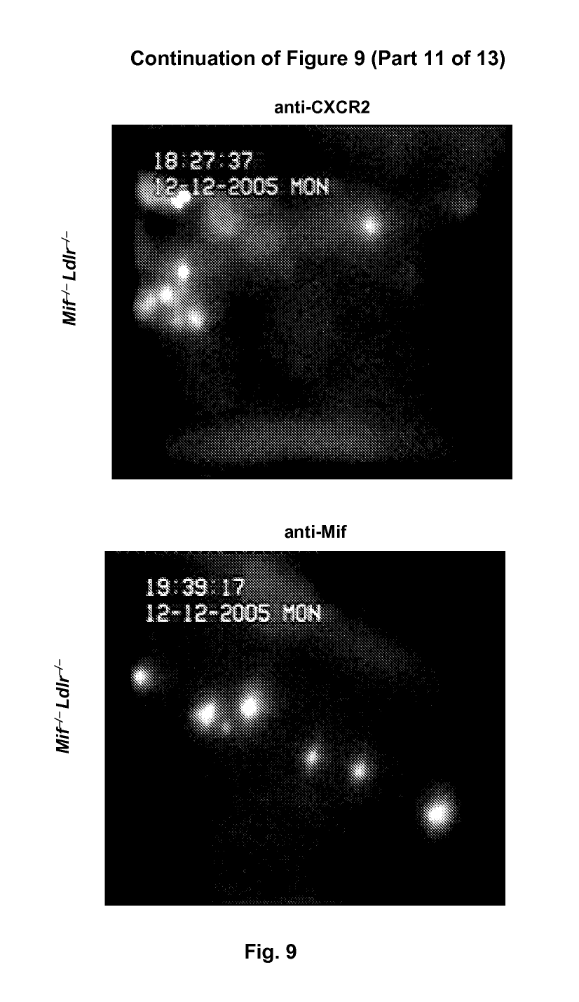

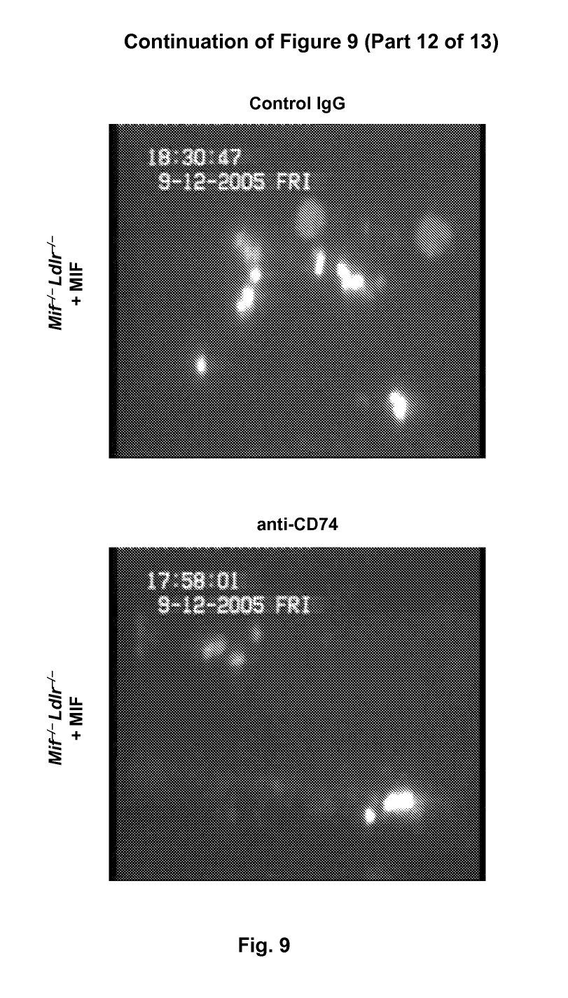

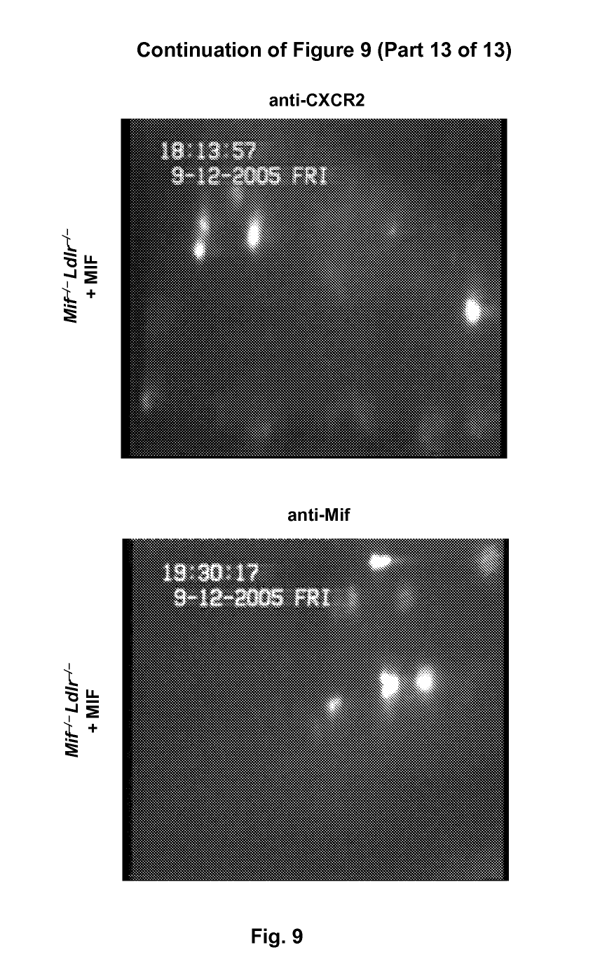

The importance of CXCR2 for MIF-mediated leukocyte recruitment under atherogenic or inflammatory conditions was corroborated in vivo. The adhesion of monocytes to the luminal surface of aortic roots was reduced in Mif^{-/-} Ldlr^{-/-} versus Mif^{+/+} Ldlr^{-/-} mice with primary atherosclerosis, and this was mirrored by a marked decrease in lesional macrophage content (FIG. 6a). Intravital microscopy of microcirculation in the cremaster muscle revealed that injecting MIF adjacent to the muscle caused a marked increase in (mostly CD68⁺) leukocyte adhesion and emigration in postcapillary venules (data not shown), which was inhibited by an antibody to CXCR2 (FIG. 6b,c). Circulating monocyte counts were unaffected (data not shown).

Next a model of MIF-induced peritonitis was used in chimeric mice reconstituted with wild-type or Il8rb−/− bone marrow. Intraperitoneal injection of MIF elicited neutrophil recruitment after 4 h in mice with wild-type bone marrow, which was abrogated in mice with Il8rb^{-/-} bone marrow (FIG. 6d). Collectively, these results demonstrated that MIF triggers leukocyte recruitment under atherogenic and inflammatory conditions in vivo through CXCR2.

### Targeting MIF Results in Regression of Atherosclerosis

As described herein, MIF acted through both CXCR2 and CXCR4. Given the role of MIF and CXCR2 in the development of atherosclerotic lesions, targeting MIF, rather than CXCL1 or CXCL12, was investigated as a method to modify advanced lesions and their content of CXCR2⁺ monocytes and CXCR4⁺ T cells. Apoe^{-/-} mice, which had received a high-fat diet for 12 weeks and had developed severe atherosclerotic lesions, were treated with neutralizing antibodies to MIF, CXCL1 or CXCL12 for 4 weeks. Immunoblotting and adhesion assays were used to verify the specificity of the MIF antibody. These assays confirmed that the MIF antibody blocked MIF-induced, but not CXCL1- or CXCL8-induced, arrest (data not shown).

Blockade of MIF, but not CXCL1 or CXCL12, resulted in a reduced plaque area in the aortic root at 16 weeks and a significant (P<0.05) plaque regression compared to baseline at 12 weeks (FIG. 6e,f). In addition, blockade of MIF, but not CXCL1 or CXCL12, was associated with less of an inflammatory plaque phenotype at 16 weeks, as evidenced by a lower content of both macrophages and CD3⁺ T cells (FIG. 6g,h). Therefore, by targeting MIF and inhibiting the activation of CXCR2 and CXCR4, therapeutic regression and stabilization of advanced atherosclerotic lesions was achieved.

From the foregoing, it will be obvious to those skilled in the art that various modifications in the above-described methods, and compositions are made without departing from the spirit and scope of the invention. Accordingly, in some embodiments the invention is embodied in other specific forms without departing from the spirit or essential characteristics thereof. Present embodiments and examples, therefore, are to be considered in all respects as illustrative and not restrictive, and all changes which come within the meaning and range of equivalency of the claims are therefore intended to be embraced therein.

What is claimed is:

1. A method of treating atherosclerosis in a patient in need thereof, comprising administering to the patient one or more agents that, either individually or in combination, inhibit: (i) MIF-binding to CXCR2 and CXCR4 and/or (ii) MIF-activation of CXCR2 and CXCR4; or (iii) any combination of (i) and (ii).

2. A method of inhibiting atherogenic cell recruitment in a patient in need thereof; comprising administering to the patient one or more agents that inhibit MIF-binding to CXCR2 and CXCR4 and/or MIF-activation of CXCR2 and CXCR4.

3. A method of inhibiting inflammatory cell recruitment to atherosclerotic lesions in a patient in need thereof, comprising administering to the patient one or more agents that inhibit MIF-binding to CXCR2 and CXCR4 and/or MIF-activation of CXCR2 and CXCR4.

4. A method of inducing regression of pre-existing atherosclerotic plaques or inducing a stable plaque phenotype in a patient in need thereof, comprising administering to the patient one or more agents that inhibit MIF-binding to CXCR2 and CXCR4 and/or MIF-activation of CXCR2 and CXCR4.

5. A method of reducing macrophage and T-cell content of an atherosclerotic plaque in a patient in need thereof, comprising administering to the patient one or more agents that inhibit MIF-activation of CXCR2 and CXCR4 and/or MIF-binding to CXCR2 and CXCR4.

6. The method of any of claims 1-5, further comprising administering an agent that inhibits MIF-activation of CD74 and/or MIF-binding to CD74.

7. The method of any of claims 1-5, wherein the agent is an antibody that inhibits MIF-activation of CXCR2 and CXCR4.

8. The method of any of claims 1-5, wherein a first agent is a first antibody that inhibits MIF-activation of CXCR2 and a second agent is a second antibody that inhibits MIF-activation of CXCR4.

9. The method of any of claims 1-5, wherein the agent is an antibody that inhibits MIF-binding to CXCR2 and CXCR4.

10. The method of any of claims 1-5, wherein a first agent is a first antibody that inhibits MIF-binding to CXCR2 and a second agent is a second antibody that inhibits MIF-binding to CXCR4.

11. The method of any of claims 1-5, wherein the agent is an antibody that binds to MIF.

12. The method of claim 11, wherein the antibody is selected from a chimeric, humanized, human, bi-specific, or grafted antibody.

13. The method of claims 11, wherein the antibody is a human or humanized antibody.

14. The method of any of claims 1-5, wherein the agent is an antigen binding fragment.

15. The method of claim 14, wherein the antigen binding fragment is selected from F(ab′)₂, Fab′, Fab, Fv, scFv, Fd, Fd′, V_{L}, V_{H}, a single chain binding polypeptide, a bispecific fragment, a diabody, a bivalent scFv, and a tetrameric scFv.

16. The method of any of claims 1-5, wherein one agent is a CXCR4 antagonist.

17. The method of claim 16, wherein the CXCR4 antagonist is a small molecule.

18. The method of claim 17, wherein the small molecule antagonist of CXCR4 is selected from ALX40-4C, AMD-070, AMD3100, AMD3465, KRH-1636, KRH-2731, KRH-3955, KRH-3140, T134, T22, T140, TC14012, TN14003, RCP168, POL3026, and CTCE-0214.

19. The method of any of claims 1-5, wherein at least one agent specifically binds the pseudo-ELR motif of MIF or interferes with the binding of the pseudo-ELR motif of MIF to its targets.

20. The method of any of claims 1-5, wherein at least one agent comprises a peptide or peptide mimetic of MIF, CXCR2, CXCR4, or CD74.

21. A composition comprising an agent that inhibits MIF-binding to CXCR2 and CXCR4 and/or MIF-activation of CXCR2 and CXCR4.

22. The composition of claim 21, in which the agent inhibits MIF-activation of CD74 and/or MIF-binding to CD74.

23. The composition of claim 21, in which the agent is an antibody that inhibits MIF-activation of CXCR2 and CXCR4.

24. The composition of claim 21, in which the agent is an antibody that inhibits MIF-binding to CXCR2 and CXCR4.

25. The composition of claim 21, in which the agent is an antibody that binds to MIF.

26. The composition of claim 25, in which the antibody is selected from a chimeric, humanized, human, bi-specific, or grafted antibody.

27. The composition of claim 25, in which the antibody is a human or humanized antibody.

28. The composition of claim 21, in which the agent is an antigen binding fragment.

29. The composition of claim 28, in which the antigen binding fragment is selected from F(ab′)₂, Fab′, Fab, Fv, scFv, Fd, Fd′, V_{L}, V_{H}, a single chain binding polypeptide, a bispecific fragment, a diabody, a bivalent scFv, and a tetrameric scFv.

30. The composition of claim 21, in which the agent is a CXCR4 antagonist.

31. The composition of claim 21, in which the agent is a CXCR2 antagonist.

32. The composition of claim 21, in which the agent specifically binds the pseudo-ELR motif of MIF or interferes with the binding of the pseudo-ELR motif of MIF to its targets.

33. The composition of claim 21, in which the agent comprises a peptide or peptide mimetic of MIF, CXCR2, CXCR4, or CD74.

34. A pharmaceutical composition comprising at least one agent in any of claims 21-33.
